(19) United States
(12) Patent Application Publication
(10) Pub. No.: US 2010/0094113 A1
(43) Pub. Date: Apr. 15, 2010

(54) HEMODYNAMIC MONITORING DURING AUTOMATED MEASUREMENT OF BLOOD CONSTITUENTS

(76) Inventors: Mark Ries Robinson, Albuquerque, NM (US); Mike Borrello, Carlsbad, CA (US); William R. Patterson, Irvine, CA (US)

Correspondence Address: V. Gerald Grafe, esq., P.O. Box 2689, Corrales, NM, 87048, US

(21) Appl. No.: 12/576,121

(22) Filed: Oct. 8, 2009

Related U.S. Application Data
(60) Provisional application No. 61/104,193, filed on Oct. 9, 2008.

(51) Int. Cl. A61B 5/1473; A61B 5/02; A61B 5/021

(52) U.S. Cl. 600/345; 600/485; 600/483

(57) ABSTRACT

The present invention provides methods and apparatuses that can provide measurement of analytes such as glucose with a variety of sensors in connection with hemodynamic monitoring. Some embodiments of the present invention enable the use of a single arterial access site for automated blood glucose measurement as well as hemodynamic monitoring. Some embodiments of the present invention can reduce or eliminate nuisance hemodynamic alarms. Some embodiments of the present invention can provide hemodynamic monitoring during an automated analyte measurement process. An example apparatus according to the present invention comprises a blood access system, adapted to remove blood from a body and infuse at least a portion of the blood back into the body. Such an apparatus also comprises an analyte sensor, mounted with or integrated into the blood access system such that the analyte sensor measures the analyte in the blood that has been removed from the body by the blood access system.

### CROSS REFERENCE TO RELATED APPLICATIONS

This application claims priority to U.S. provisional application 61/104193, filed Oct. 9, 2008, which is incorporated herein by reference. This application is related to the following patent applications, each of which is incorporated herein by reference:
U.S. provisional 60/791,719, filed Apr. 12, 2006;
U.S. provisional 60/913,582, filed Apr. 24, 2007;
PCT application PCT/US06/60850, filed Nov. 13, 2006;
U.S. application Ser. No. 11/679,826, filed Feb. 27, 2007;
U.S. application Ser. No. 11/679,837, filed Feb. 28, 2007;
U.S. application Ser. No. 11/679,839, filed Feb. 28, 2007;
U.S. application Ser. No. 11/679,835, filed Feb. 27, 2007;
U.S. application Ser. No. 10/850,646, filed May 21, 2004;
U.S. application Ser. No. 11/842,624, filed Aug. 21, 2007;
U.S. application Ser. No. 12/188205, filed Aug. 8, 2008;
U.S. provisional 60/991,373, filed Nov. 30, 2007;
U.S. provisional 61/044,004, filed Apr. 10, 2008;
U.S. application Ser. No. 12/108,250 Apr. 23, 2008.

### BACKGROUND

Since 2001, a number of intensive care units have adopted tight glycemic control protocols for the maintenance of glucose at close to physiological levels. The process of maintaining tight glycemic control requires frequent blood glucose measurements. The blood utilized for these measurements is typically obtained by procurement of a sample from a fingerstick, arterial line, or central venous catheter. Fingerstick measurements are generally considered undesirable due to the pain associated with the fingerstick process and the nuisance associated with procurement of a quality sample. Sample procurement from central venous catheters can also present problems since current clinical protocols recommend the stoppage of all fluid infusions prior to the procurement of a sample. Consequently, the use of arterial catheters has become more common. Arterial catheters are typically placed for hemodynamic monitoring of the patient and provide real-time continuous blood pressure measurements. These catheters are maintained for a period of time and used for both hemodynamic monitoring and blood sample procurement. Arterial catheters are not typically used for drug or intravenous feedings so issues associated with cross-contamination are minimized.

The process of procuring an arterial blood sample for measurement typically involves the following steps. The slow saline infusion used to keep the artery open is stopped and some type of valve mechanism such as a stopcock is opened to allow fluid connectivity to the mechanism for blood draw. The process of opening the stopcock and concurrently closing off fluid connectivity to the pressure transducer will cause a stoppage of patient pressure monitoring as the transducer no longer has direct fluid access to the patient. The sample procurement process is initiated. The initial volume drawn through the stopcock is saline followed by a transition period of blood and saline and subsequently pure blood. Generally, at the point where there is no or very little saline in the blood sample at the stopcock (or a knowable saline concentration), the measurement sample is obtained. The blood and saline sample obtained previously can be discarded or infused back into the patient.

In many intensive care units, a significant portion of blood samples obtained from arterial catheters are procured using blood sparing systems. In this process a leading sample containing both saline and blood is withdrawn from the patient and stored in a reservoir that lies beyond the sample acquisition port. A sample of blood that is free of saline contamination can then be procured at the sample port for measurement. Example embodiments of such blood sparing techniques include the Edward's VAMP system, shown in FIG. 1, and the Abbott SafeSet system. The Edward's VAMP in-service poster is incorporated by reference. Following procurement of an undiluted sample for measurement, the remaining blood/saline mixture can be re-infused into the patient.

Hemodynamic pressure monitoring is unavailable during the procurement of the blood sample by either the syringe method or by use of a blood sparing system. If the standard stopcock is replaced with a 4-way stopcock it would allow the transducer and the blood sampling system to be in fluid connectivity with the patient. In such a situation the withdrawal process creates a pressure gradient that will limit the accuracy of the existing hemodynamic monitoring system.

The development of an automated blood glucose measurement system for use in the intensive care unit is highly desired due to reductions in labor, increased measurement frequency, and an improved ability to limit potentially dangerous conditions of hypoglycemia. The ability to attach such a system to an arterial access site is desired as catheter patency for blood sample procurement is typically better at an arterial access location than at a venous access site. As placement of an arterial catheter is considered a moderately invasive procedure, it is undesirable to require placement of two such catheters, one used for pressure monitoring and another for blood access. Thus, in clinical practice it is desirable to use one arterial access site for both hemodynamic monitoring as well as a blood access site for automated glucose measurement. Such sharing of a single site can result in hemodynamic monitoring disruption during the blood procurement process. For example, if the automated blood measurement system acquires a sample every 15 minutes, it will likely interfere with the hemodynamic pressure monitoring system so as to cause an alarm or produce inaccurate pressure measurements. The management of such an alarm typically requires nurse intervention, defeating some of the advantages sought with an automated blood measurement system. In addition to nuisance alarms, the real-time hemodynamic monitoring may be disrupted during the automated measurement process. In those patients that are hemodynamically unstable, such a disruption may be an unacceptable consequence of automated blood glucose monitoring.

### SUMMARY OF THE INVENTION

The present invention comprises methods and apparatuses that can provide measurement of glucose and other analytes with a variety of sensors in connection with hemodynamic monitoring. Some embodiments of the present invention enable the use of a single arterial access site for automated blood glucose measurement as well as hemodynamic monitoring. Most arterial catheter are peripherally placed arterial catheters but this specification specifically includes any catheter placed in an arterial vessel including, as examples, femoral arteries, ulnar arteries, radial arteries, and the pulmonary artery. Some embodiments of the present invention can reduce or eliminate nuisance hemodynamic alarms. Some embodiments of the present invention can provide hemodynamic monitoring during an automated analyte measurement process. An example apparatus according to the present invention comprises a blood access system, adapted to remove blood from a body and infuse at least a portion of the blood back into the body. Such an apparatus also comprises an analyte sensor, mounted with or integrated into the blood access system such that the analyte sensor measures the analyte in the blood that has been removed from the body by the blood access system.

A method according to the present invention comprises making such an automated analyte measurement while minimizing the impact on hemodynamic monitoring from an arterial catheter. The description herein will use an example blood access system for convenience. Other blood access systems and other analyte measurement techniques are also suitable for use with the present invention, as examples including those described in the patent applications incorporated by reference herein.

Some embodiments of the present invention enable attachment of an automated glucose measurement system to an existing hemodynamic monitoring system while maintaining the necessary dynamic response of the hemodynamic monitoring system for accurate blood pressure measurement.

Some embodiments of the present invention enable incorporation of an automated glucose measurement system into an existing hemodynamic monitoring system while maintaining the necessary dynamic response for accurate blood pressure measurement.

Some embodiments of the present invention can provide automated analyte measurements while concurrently providing hemodynamic monitoring during the measurement process.

Some embodiments of the present invention create an artificial or surrogate waveform during periods of hemodynamic disruption. The use of the surrogate waveform prevents the triggering of hemodynamic alarms and is readily identifiable by the clinician as a surrogate waveform.

Some embodiments of the present invention can provide automated analyte measurements while minimizing the error imparted to the hemodynamic monitoring system by using pressure gradients that reduce measurement errors to levels that are below a clinically significant threshold.

Some embodiments of the present invention utilize a single arterial access site but utilize a dual lumen catheter so as to minimize hemodynamic monitoring disruption by an automated blood measurement system.

### BRIEF DESCRIPTION OF DRAWINGS

The accompanying drawings, which are incorporated in and form part of the specification, illustrate the present invention and, together with the remainder of the application and information available to those of ordinary skill in the art, describe the invention.

FIG. 1 is an example of a blood sparing device.

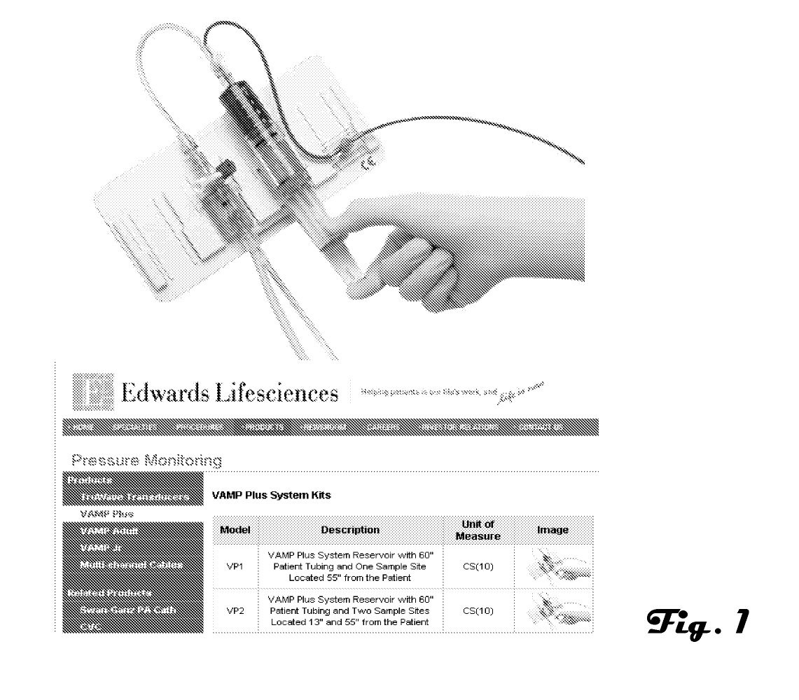

FIG. 2 is an graphical representation of Gardner's criteria, often refered to as Gardner's wedge.

FIG. 3 is an example of a standard arterial catheter pressure monitoring configuration.

FIG. 4 is an example of an automated blood analyte system attached to an arterial pressure monitoring system.

FIG. 5 is an example configuration which enables creation of a surrogate pressure trace.

FIG. 6 is an example of an actual pressure trace and a surrogate signal trace.

FIG. 7 is an example of an actual pressure trace and a surrogate signal trace.

FIG. 8 is an example of an automated blood analyte monitoring circuit.

FIG. 9 is an example of a blood access system that enables concurrent pressure monitoring.

FIG. 10 is an example of a blood access system where the sensor is located near the patient.

FIG. 11 is a block diagram showing the key components of the model estimation process.

FIG. 12 is a model of the blood access system.

FIG. 13 is an example demonstration of the equations used to provide concurrent pressure monitoring during the withdrawal sequence.

FIG. 14 is an example display of an automated blood analyte system.

FIG. 15 is a diagram showing the system used to create an artificial patient with a variable pressure, variable volume chamber.

FIG. 16 shows the test configuration used for accessing pressure differences.

FIG. 17 shows a test waveform.

FIG. 18 shows results Bode plot of several test configurations.

FIG. 19 shows a waveform test result from several test configurations.

FIG. 20 shows the hemodynamic monitoring errors introduced by a measurement cycle.

FIG. 21 shows the various flow types used in a measurement cycle.

FIG. 22 shows the periods during which hemodynamic monitoring information has a potential error.

FIG. 23 is a summary table of the errors generated during testing as a function of flow type.

FIG. 24 is an illustration of the waveform results from a representative flow type.

FIG. 25 is an illustration of the waveform results from a representative flow type.

FIG. 26 is an illustration of the waveform results from a representative flow type.

FIG. 27 is an illustration of the waveform results from a representative flow type.

FIG. 28 is an illustration of the waveform results from a representative flow type.

FIG. 29 is an illustration of the waveform results from a representative flow type.

FIG. 30 is an illustration of the waveform results from a representative flow type.

FIG. 31 is an illustration of the key components of a dual access system using a sheath and catheter.

FIG. 32 is a illustration of a dual access system using a sheath and catheter as it relates to a patient artery.

FIG. 33 is a block diagram showing the key components of a preferred embodiment.

FIG. 34 is a block diagram showing the key components of a preferred embodiment.

FIG. 35 is a block diagram showing the key components of a preferred embodiment.

FIG. 36 is a block diagram showing the key components of a preferred embodiment.

FIG. 37 is a block diagram showing the key components of a preferred embodiment.

FIG. 38 is a block diagram showing the key components of a preferred embodiment.

FIG. 39 is a block diagram showing the key components of a preferred embodiment.

FIG. 40 is a block diagram showing the key components of a preferred embodiment.

### DETAILED DESCRIPTION OF THE INVENTION

The sharing of a single arterial access site for both hemodynamic monitoring as well as blood sample procurement requires attention to a variety of implementation details. In simple terms the automated measurement system should not: (1) change or influence the dynamic response of the hemodynamic monitoring system; (2) create pressure gradients that result in inaccurate measurements; or (3) introduce bubbles. Any of the above may create a situation where the hemodynamic values are inaccurate.

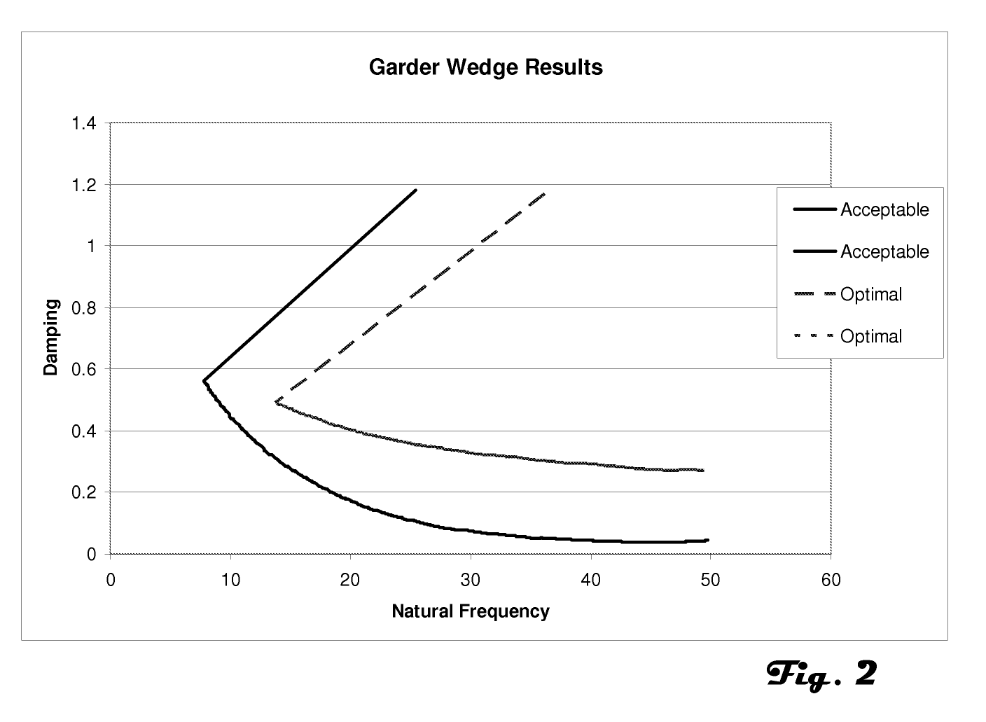

In practice the automated blood glucose measurement system should not decrease the measurement performance or accuracy of the hemodynamic monitoring system. In clinical use, a pressure monitoring system should be able accurately measure both systolic and diastolic pressures and quickly detect pressure changes. The response capability of a hemodynamic monitoring system can be defined in terms of the “frequency response” of the system and the “damping” of the system. Rapid response is needed at high heart rates or with hyperdynamic hearts. During these conditions it is essential that the system have a high “natural” frequency response. For accurate measurements, the frequency response must be matched with an appropriate degree of damping. The relationship of frequency and damping coefficient have been explored and defined by Reed Gardner. This relationship is well described in “Direct Blood Pressure Measurements—Dynamic Response Requirements” Anesthesiology pages 227-236, 1981, which is incorporated herein by reference. FIG. 2 shows the resulting relationship between damping and natural frequency. If a single access site is used for both sample procurement and pressure monitoring, the system associated with sample procurement and measurement should minimize alterations to the dynamic response of the pressure monitoring system and maintain a relationship between damping and natural frequency that satisfies the Gardner wedge requirements.

The process of completing a measurement cycle requires the development of a pressure gradient to allow flow. The measurement cycle is simply defined as any activity associated with making an automated measurement. Actions included as part of the measurement cycle include but are not limited to sample withdrawal, sample infusion, calibration, cleaning of the circuit, catheter flushing, and low-flow infusion to keep the access site open. The above actions require flow and thus necessitate a pressure gradient. This pressure gradient will influence the pressure measured by the pressure transducer. The present invention provides a blood measurement system that can minimize the degree of hemodynamic monitoring disruption. Minimization can be in terms of the magnitude of the pressure measurement error or the duration of disruption or both.

From a practical use standpoint, the nursing staff will be especially sensitive to errors in monitoring that are not easily identified as being false or nuisance alarms. A nuisance alarm is a general term applied to an alarm that occurs due to a disruption in measured pressure due to the automated measurement when in fact the actual system is not compromised and the patient condition has not deteriorated. If the blood measurement system does disrupt pressure monitoring to a clinically significant level, then the total time disruption period should be minimized or segmented into increments that minimize clinical impact. During such a disruption, the nurse should be able to easily identify that the measurements are inaccurate.

An additional requirement when using a single access site relates to the desire that the performance of the system be consistent over time. In typical clinical practice, the nurse will establish the overall performance characteristics of the hemodynamic monitoring system during the setup phase. Thus, the process of procuring a blood sample for measurement that results in the degradation of the system such that in accurate results are displayed is undesirable. For example, following a measurement, pressure monitoring errors can results for air bubbles or clots being introduced into the system. The introduction of an air bubble is especially problematic as it will alter the overall dynamic response of the system such that the previously discussed Gardener's wedge criteria are not satisfied. Such a development is especially problematic as it is not easily detectable.

### General Description of Characteristics of the Invention

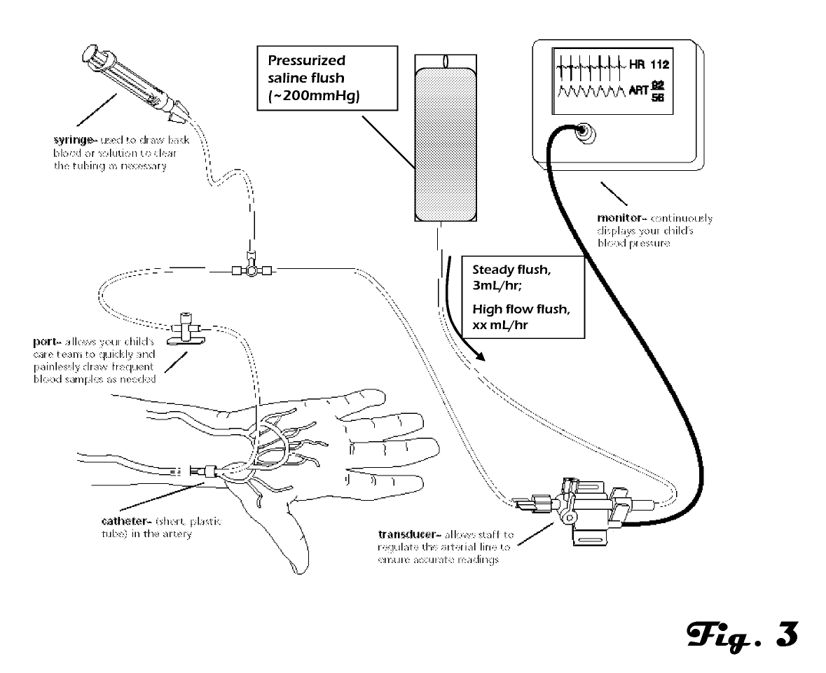

A conventional ICU pressure monitoring application is illustrated in FIG. 3. A pressure transducer is in direct fluid contact with the arterial blood through the fluid stream in the connecting tubing. In typical operation, a pressurized saline bag is used to establish a pressure gradient between the saline and the patient's arterial vasculature, resulting in infusion of a small amount of saline into the patient at a constant rate. This saline infusion helps to keep the access site open. During a typical blood withdrawal sequence, the stopcock at the pressure transducer is closed and a sample is procured by a syringe attached to the arterial catheter. During this period of time no hemodynamic monitoring occurs. Following completion of the blood sample procurement, the stopcock is again opened and hemodynamic monitoring is reinitiated.

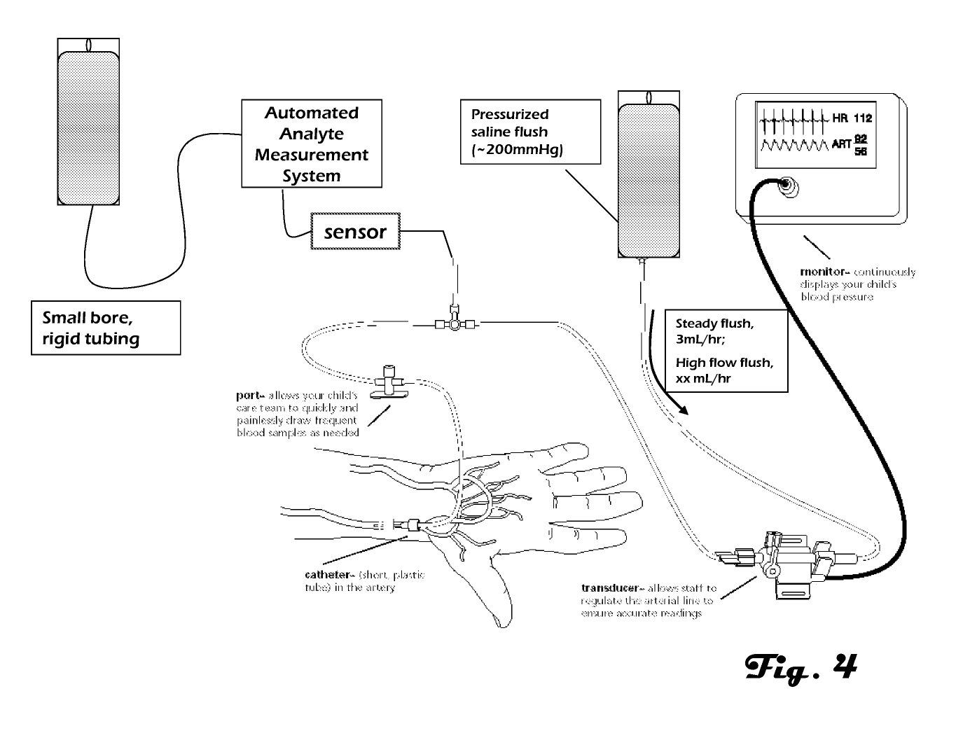

As shown in FIG. 4 an automated sample acquisition and measurement system can be attached in a similar manner. If a stopcock creating a T-junction (typically referred to as a 4-way stopcock) is used then the effects on the hemodynamic trace can be significant. The attachment of the automated blood measurement system can alter the overall response characteristics of the system such that accurate pressure measurements cannot be obtained. Assuming that simple attachment does not alter the overall dynamic response characteristics of the system, the accuracy or error of the measured pressure will be related to the pressure gradient created during sample measurement by the automated blood measurement system. In some cases the, effects on the measured pressure can be significant enough to trigger preset alarms that are in place to alert the clinician if the patient's hemodynamic system is undergoing failure. Most hemodynamic alarm systems have a minimum pulse pressure as well as a minimum diastolic pressure. The influence of the automated blood measurement system can be mitigated by closing the stopcock before each measurement. However this creates another problem as each measurement is not sufficiently automated due to the need for manual intervention with each sample.

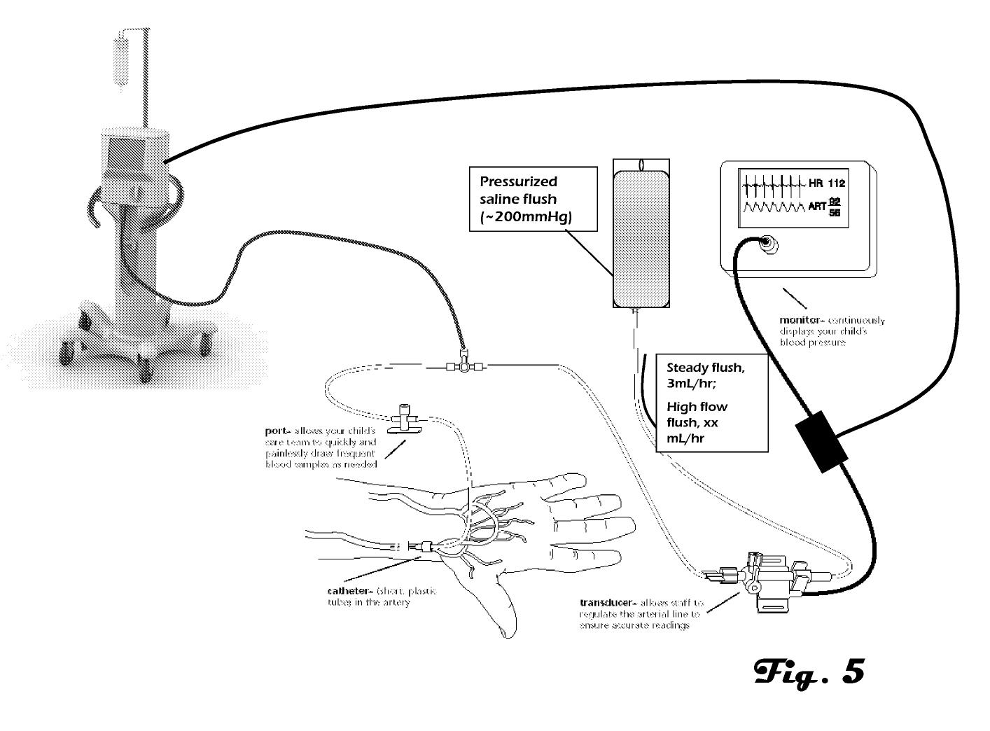

FIG. 5 is a schematic illustration of an example embodiment that addresses the monitoring problems discussed above. In the example embodiment shown, an automated blood glucose monitoring system has the ability to alter, replace or override the signal being delivered from the pressure transducer to the hemodynamic display. The resulting signal will be referred to as a surrogate signal. In FIG. 5, this is shown as a physical connection to the cable between the hemodynamic monitoring system and the pressure transducer. The communication or transfer of information between these two systems can be provided by many embodiments including, as examples, wireless communication or other communication means. An alternative embodiment has a cable from the transducer going to the automated blood measurement system and then a separate cable going from the automated blood measurement system directly to the hemodynamic display. During the period of time that the automated blood analyte measurement causes a disruption of the hemodynamic trace, the signal display on the hemodynamic monitor can be replaced by a surrogate signal. The surrogate signal can be similar to the prior hemodynamic trace but altered in a way that the clinician can readily determine that it is a surrogate or artificial trace. An example of such a surrogate signal is a square wave where the top of a square wave matches the systolic pressure, the bottom of the square wave matches the diastolic pressure and the frequency is the same as the prior arterial waveform. Such a surrogate signal allows the clinician to appreciate that a blood measurement is being made while simultaneously maintaining relevant information from the prior hemodynamic monitoring period. Most arterial pressure monitoring systems do not have the diagnostic capabilities to recognize such a surrogate signal and would therefore not alarm during its use. Other surrogate signals including a small square wave or sinusoidal wave or triangle wave can be applied to the prior pressure trace (or other representative, non-alarming pressure trace). As further examples, the display of either the automated blood analyte monitor or the hemodynamic monitor can be altered by an alteration in color or background of the display, display of error messages, or by a variety of other means. The change in the display can enable the clinician to know that the hemodynamic monitoring information may not be reflective of the actual conditions in the artery, and that surrogate pressure tracing is being displayed, while not altering the pressure indication in a way that triggers the hemodynamic alarms. This enables the automated blood analyte system to procure a sample without triggering a nuisance alarm which would require a nurse intervention. Such a surrogate trace can be required during withdrawal or infusion, but might not be required during an actual measurement process if the measurement process doesn't interfere with the normal operation of the pressure sensor to a clinically significant level. The need for such a surrogate trace is limited to those periods when the automated blood analyte measurement system is altering the hemodynamic trace at a significant level. In terms of the dilution due to the continuation of saline infusion via the pressurized saline bag, the rate of infusion can be communicated to the automated blood analyte measurement system and compensated for in a systematic manner.

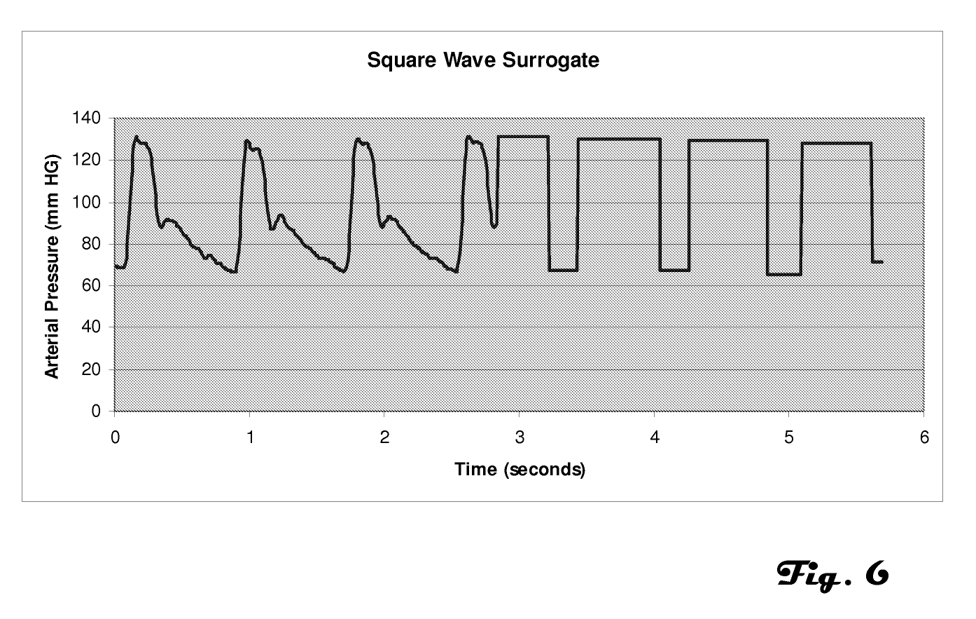

FIG. 6 shows an example of a surrogate square wave signal trace. The left-hand portion of the graph shows a true signal (reflective of the actual pressures in the artery) while the right hand portion of the graph shows a square wave with similar measurement values and frequency.

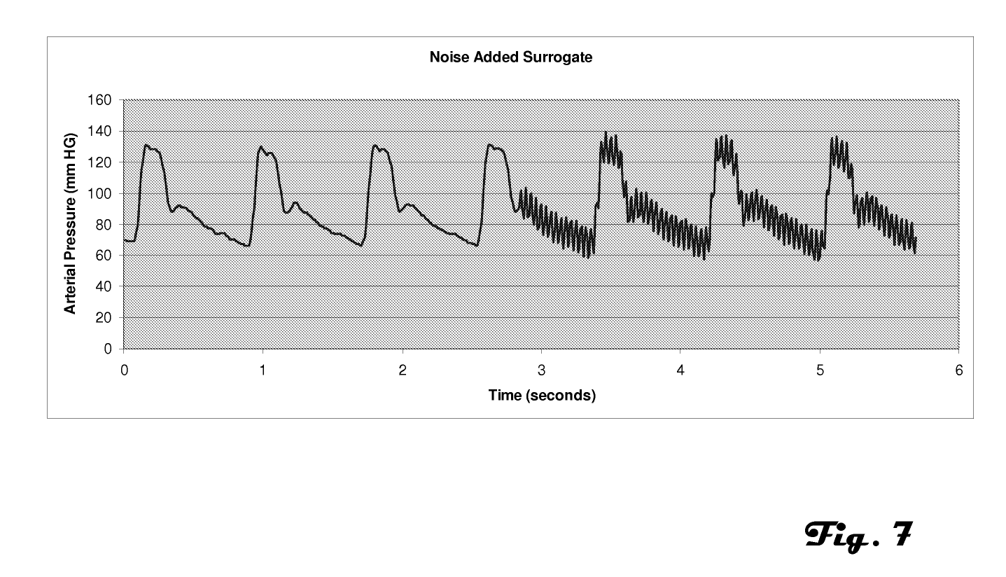

FIG. 7 shows an example of an example surrogate signal trace. The left-hand portion of the graph shows a true signal while the right hand portion of the graph shows a replication of the true signal with a noise artifact added on.

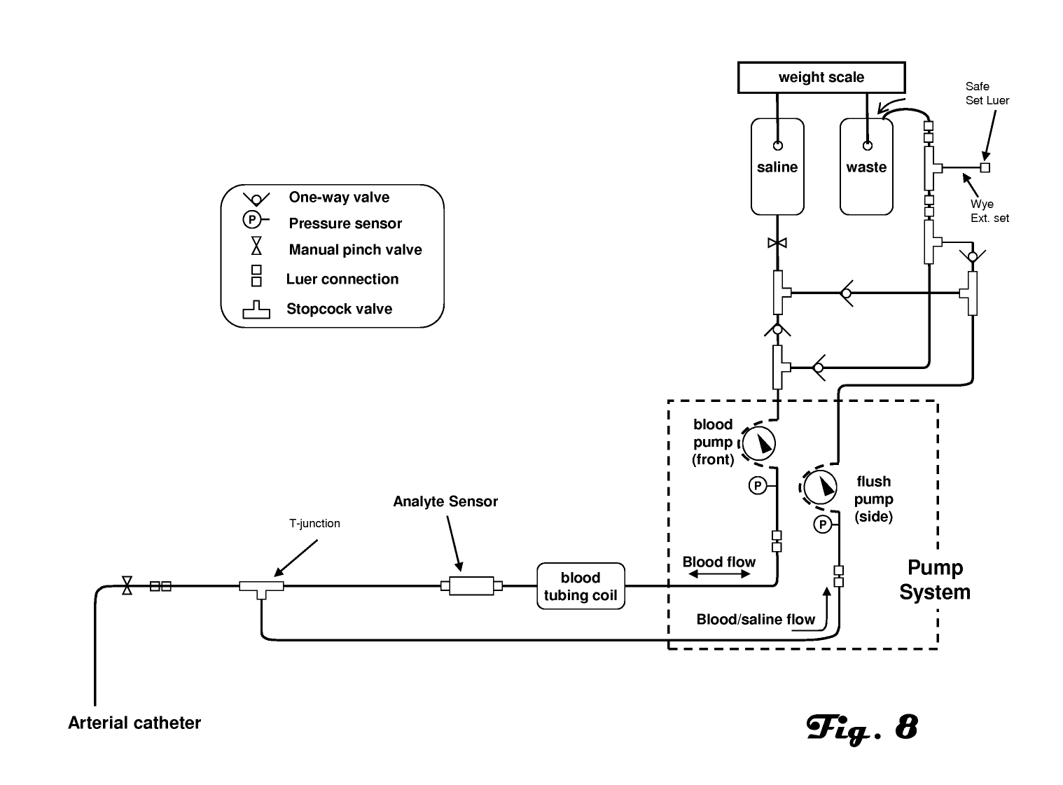

FIG. 8 is an example of an automated blood analyte measurement system. This system differs from the one illustrated in FIG. 4 in that the example system in FIG. 8 has a second tubing loop and pressure transducer that enables more effective cleaning. The blood access system shown in FIG. 8 contains two pressure transducers. During the withdrawal of blood up to the analyte sensor the pressure transducer associated with the blood pump is able to provide real-time pressure measurements associated with the blood withdrawal. During the withdrawal sequence the pressure transducer associated with the flush pump is able to effectively sense the pressure at the T-junction. The information content provided by both pressure transducers as well as the state of each blood pump can provide the basis for pressure measurement during the withdrawal sequence.

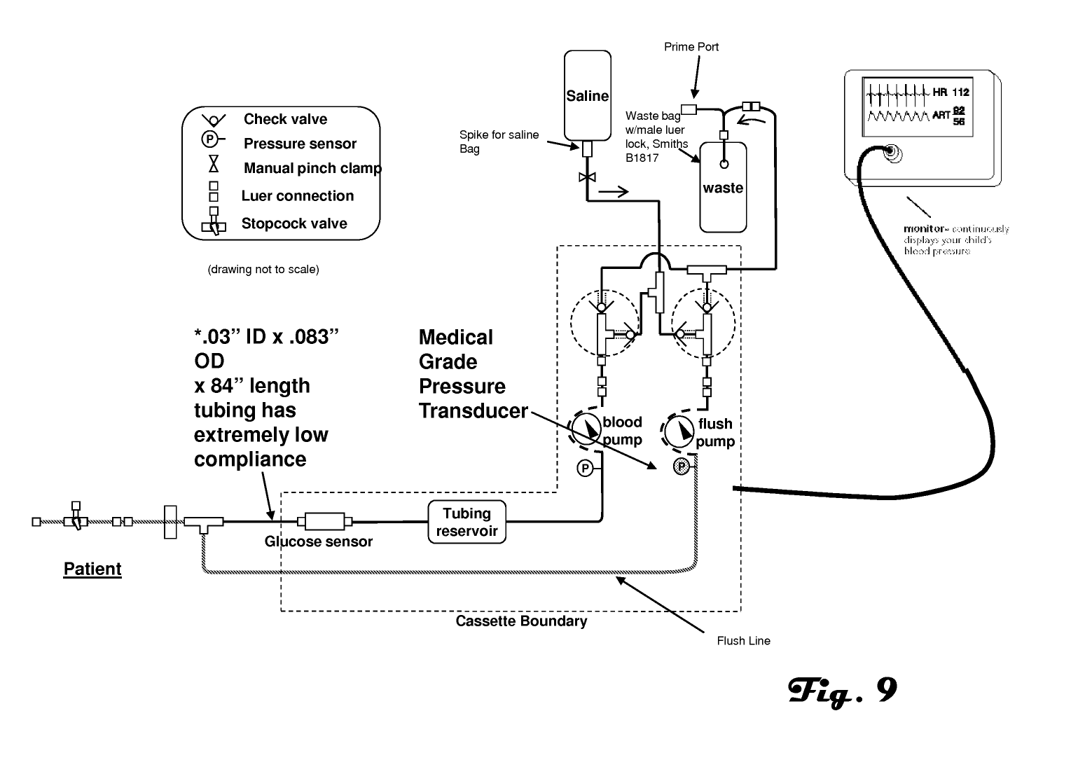

FIG. 9 is an illustration of an example embodiment of an automated blood measurement system that provides concurrent hemodynamic monitoring during the blood analyte measurement process. The automated blood withdrawal system provides a pressure signal for display on a hemodynamic monitor. In operation the blood access system is attached to the arterial catheter (not shown) and saline infused to keep the access site open is provided by the blood access system via the associated pressurized saline bag. At the time an automated blood analyte measurement is initiated, the system can stop the saline infusion into the arterial catheter and initiate a blood withdrawal process. The stoppage of flow typically present to maintain arterial access patency is desired as it enables an undiluted sample to be obtained. As the infusion rates for maintenance of catheter patency may vary by hospital, IV tubing set-up, the pressure of the bag, etc, the ability to procure an undiluted sample is an advantage of the combined system. Through the use of both pressure transducers as well as knowledge regarding the state of both pumps, the system has knowledge of the pressure artifact being created by the automated blood measurement system. These artifacts can be due to the blood withdrawal process, calibration, cleaning, infusion or fluid movement associated with the measurement cycle. Due to knowledge of the artifact created (duration, type and magnitude) the system can create a surrogate signal as described above during the period when the artifact exceeds an acceptable clinical threshold.

Instead of providing a surrogate signal, the system also has the ability to compensate for the pressure artifact being introduced by the automated blood measurement system. Through the use of both pressure transducers as well as knowledge regarding the state of both pumps, the pressure artifact can be determined enabling the determination of the true pressure at the arterial catheter. This process enables the procurement of an undiluted blood sample to the measurement system while concurrently affording real-time hemodynamic monitoring. The ability to determine the pressure gradients being produced by the automated blood measurement system enables hemodynamic monitoring to continue during a greater portion if not all of the measurement cycle. The provision of an accurate pressure trace during the entire automated analyte measurement sequence means that the patient's hemodynamic status and associated alarm methodologies remain fully operational and active during the automated blood analyte measurement.

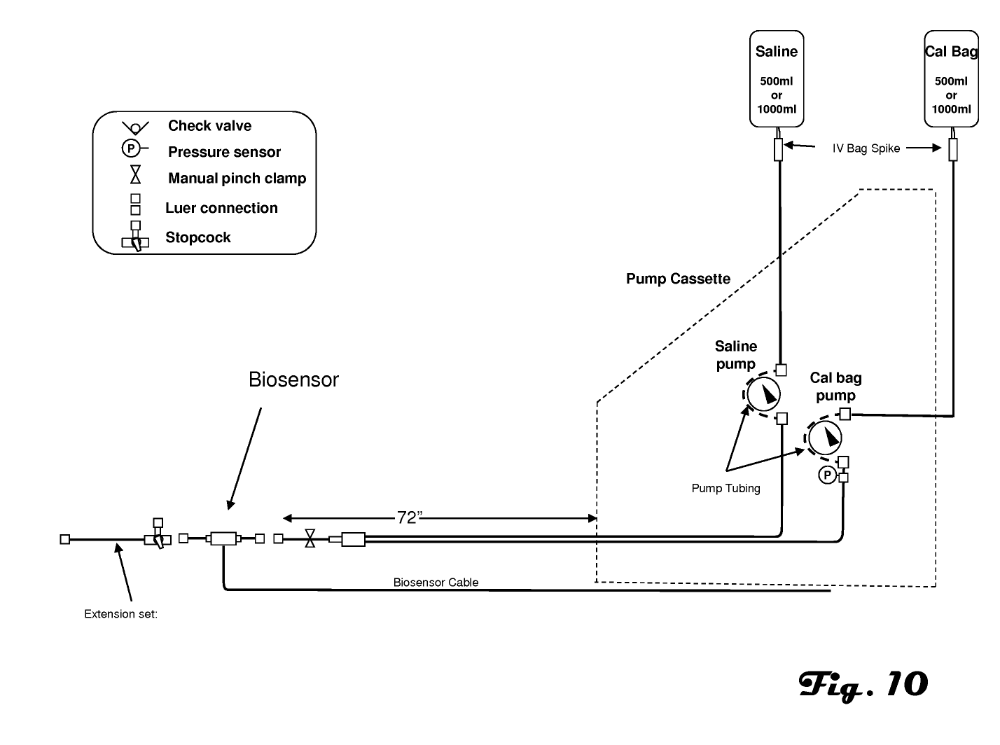

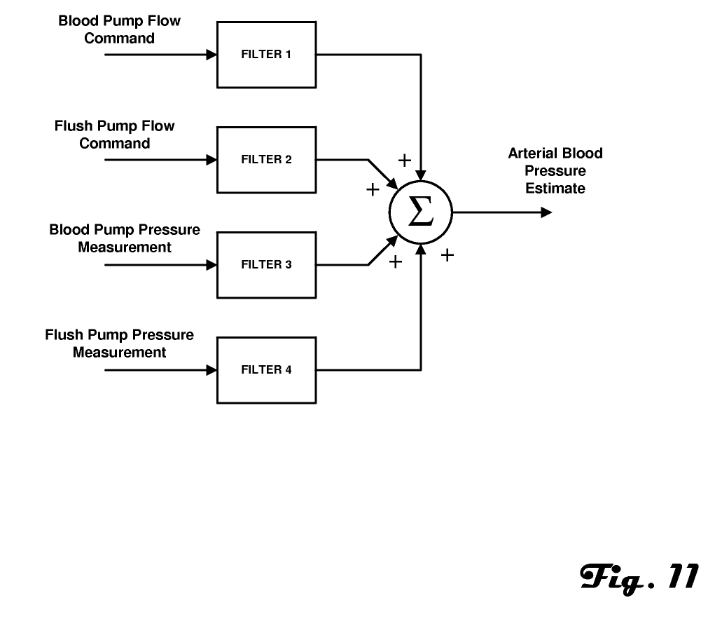

FIG. 10 shows another example embodiment of a blood access system where the sensor is located close to the patient. As shown the blood access system has only one pressure transducer but others can be added. This system with the blood sensor located more proximal to the patient also has the ability to generate surrogate signals as well as to provide direct artifact compensation. FIG. 11 shows an example of an estimator structure suitable for use with embodiments of the present invention such as that in FIG. 9. The disclosed structure enables estimation of the arterial pressure wave during the measurement process. As shown in the example estimator, the inputs to the estimator function are the blood pump flow commands, the flush pump flow commands, the blood pump pressure measurements and the flush pump pressure measurements. These commands can be utilized by a model based estimation function to provide continuous arterial blood pressure waveforms.

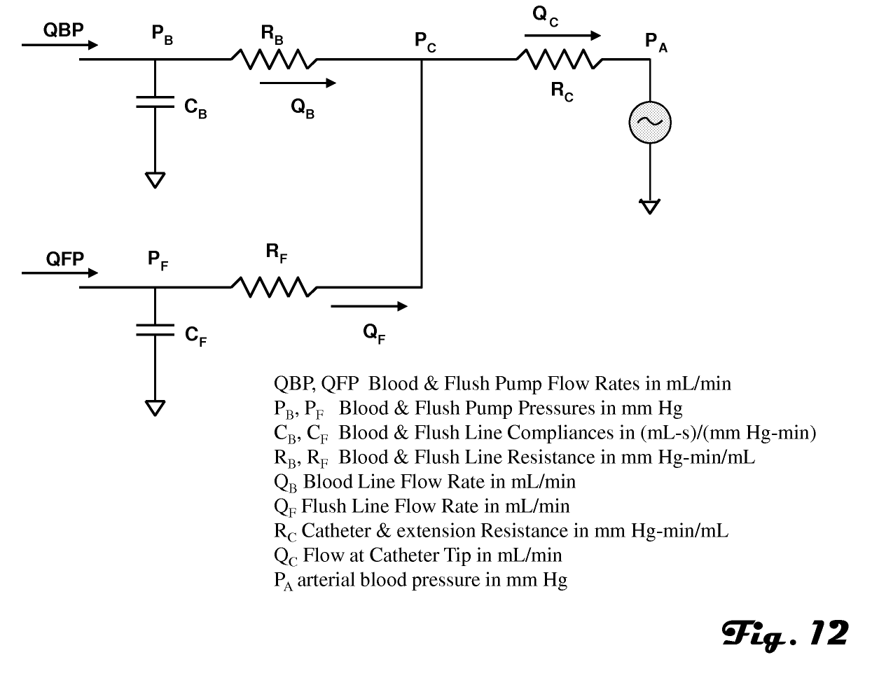

FIG. 12 shows an example method for modeling the performance of the blood access system. This model provides the basis for creating a lumped parameter linear dynamic model. The use of a linear electrical circuit analogy with multiple inputs and multiple outputs provides a basis for determining the arterial pressure during the measurement sequence. The compliances and resistances of the circuit can be accounted for in the model. The flow commands to the pump as well as the pressure measurements made can be utilized as inputs into this model to enable an estimation of the arterial pressure output. The result is a filtered linear combination of measurements and input commands for the effective estimation of the arterial pressure under any set of operational conditions.

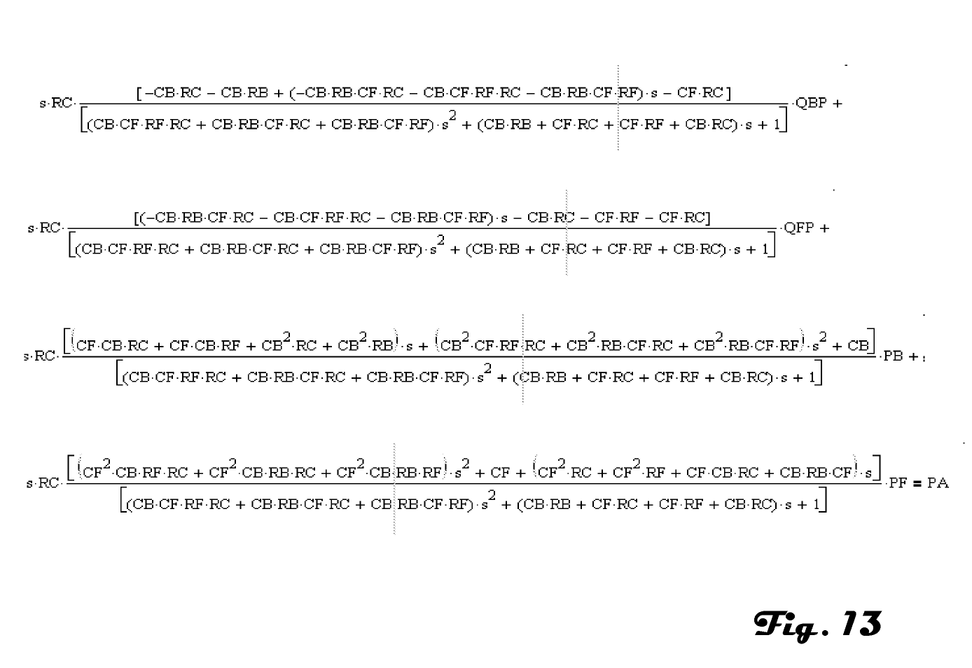

FIG. 13 is an illustration of equations that can be used to estimate the arterial pressure. As an example implementation, these equations can be programmed into the automated analyte measurement system.

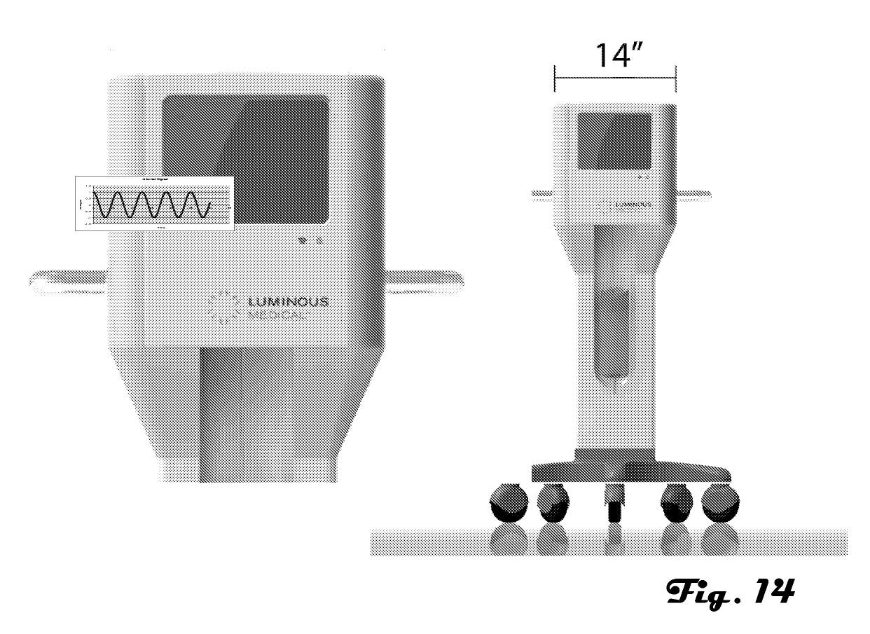

FIG. 14 is an illustration of an alternative embodiment where the arterial pressure trace or hemodynamic monitoring information is displayed on the automated blood analyte system console. In this case the automated analyte system provides analyte measurement results as well as arterial pressure measurements. The console displayed is one from Luminous Medical (a trademark of Luminous Medical, Inc.).

Although a representative blood access system has been used as an example embodiment, one of ordinary skill in the art will recognize that a variety of blood access systems provide the needed information for the effective estimation of arterial pressures during a withdrawal sequence. Specifically, the enclosed blood access system utilizes a flush circuit to facilitate cleaning of the device. Such additional tubing may not be required for the effective estimation of arterial pressures.

### Experimental Testing

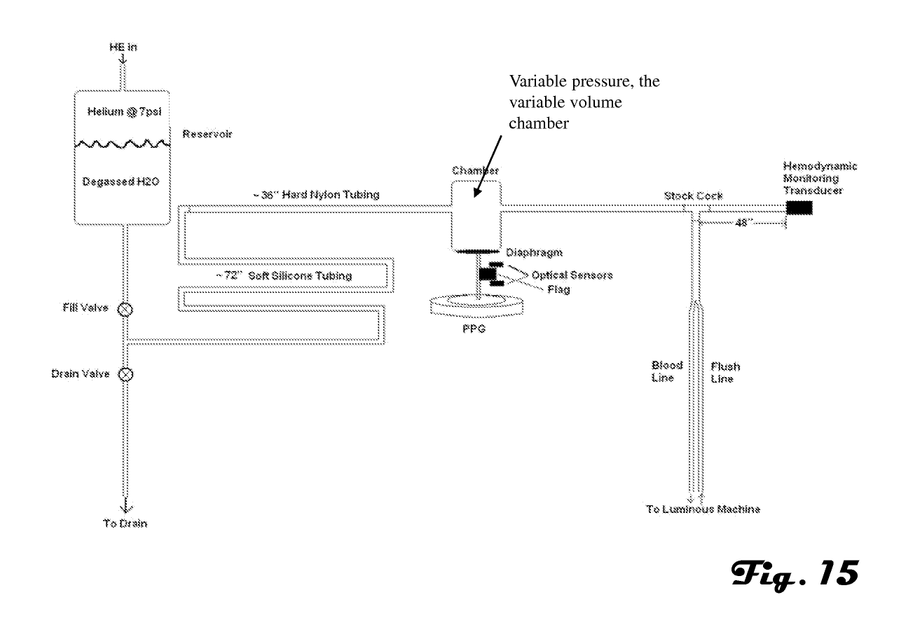

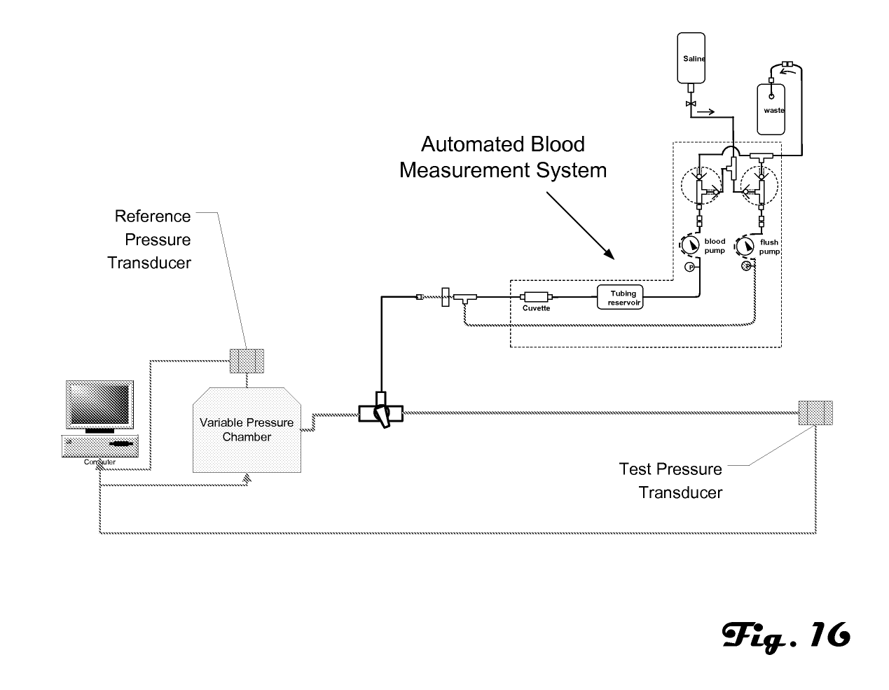

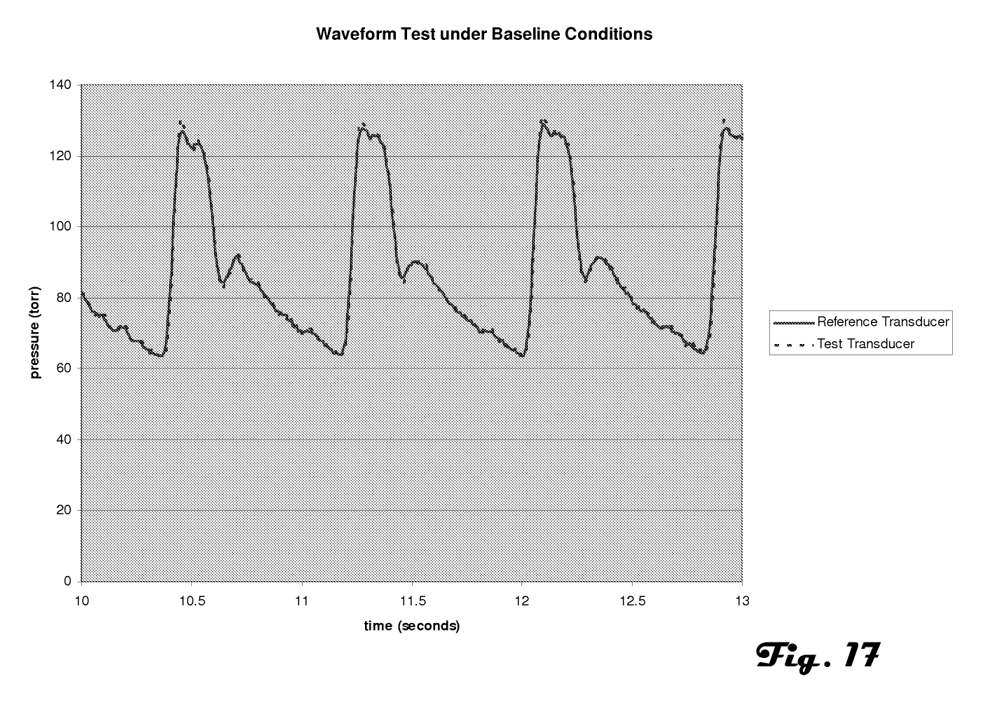

To quantify the impact of attachment of an automated system according to the present invention, like those described herein, to an existing hemodynamic monitoring system a study was conducted using a system that could simulate arterial pressure waves. The system was composed of a variable pressure, variable volume chamber (serving as an artificial patient) that could create variable pressures that matched an arterial pressure waveform under infusion and withdrawal conditions. The pressure waveforms used were obtained from a physiological database and had heart rates between 60-120 bpm with a pressure range of 150/50 mmHg. Pulse pressure generation was obtained by a diaphragm connected to a voice coil. During infusion or withdrawal, the volume of the chamber was maintained within a reasonable a range so that the pressure generation system can create accurate reproductions of arterial pressure waves. A volume control mechanism maintained the volume of the chamber so that the voice coil operated within its normal/linear range. FIG. 15 shows the overall system configuration. FIG. 16 shows the relationship between the pressure transducers under test and their relationship to the variable pressure chamber. A reference pressure transducer records the pressure generated at the artificial patient while a second test transducer records the pressures in a configuration that mirrors a conventional hemodynamic monitoring setup. Comparison between the reference and test readings enables determination of measurement errors. FIG. 17 shows an illustrative arterial pressure tracing. The agreement between the reference transducer and the test transducer is extremely good. Both pressure recordings are plotted but the level of agreement makes delineation of the two lines difficult.

Hemodynamic monitoring systems can be characterized by a variety of tests defined in the AAMI document titled “Evaluation of Clinical Systems for Invasive Blood Pressure Monitoring”, which is incorporated herein by reference. The two tests used for evaluation of performance were the sweep test and a direct comparison between reference pressure measurements at the “artificial patient” and test pressure measurements. Initial testing was conducted with only the transducer in fluid connectivity with the artificial patient. The damping and natural frequency values determined from the sweep test were acceptable when evaluated by the Gardner wedge. FIG. 2 shows the Gardner wedge. Waveform testing demonstrated an average difference of less than 1% when comparing the pressure waveform recorded at the reference or “artificial patient” and the pressure waveform recorded at the test transducer.

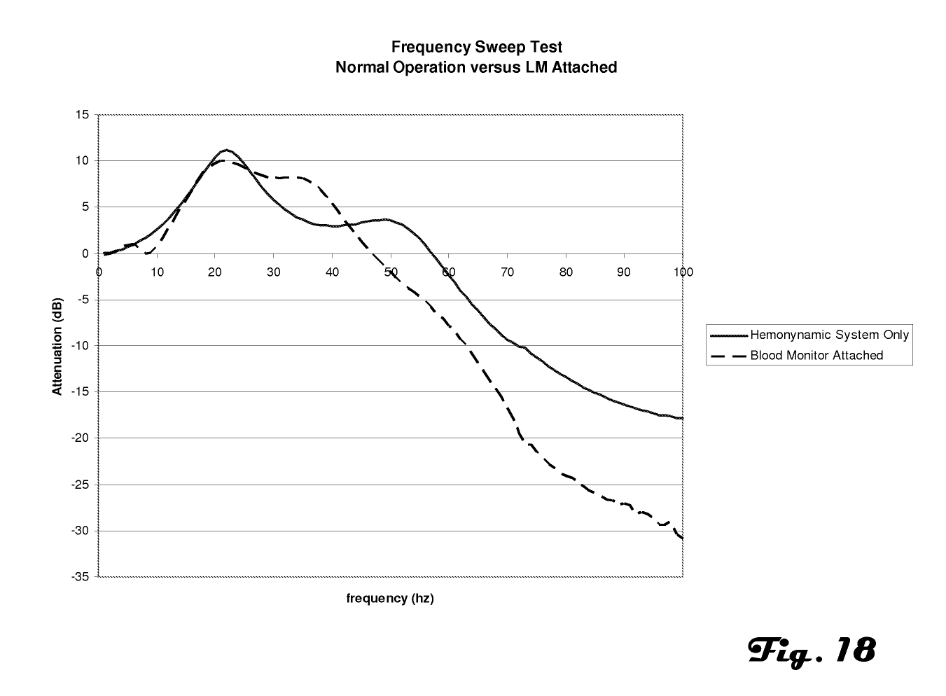

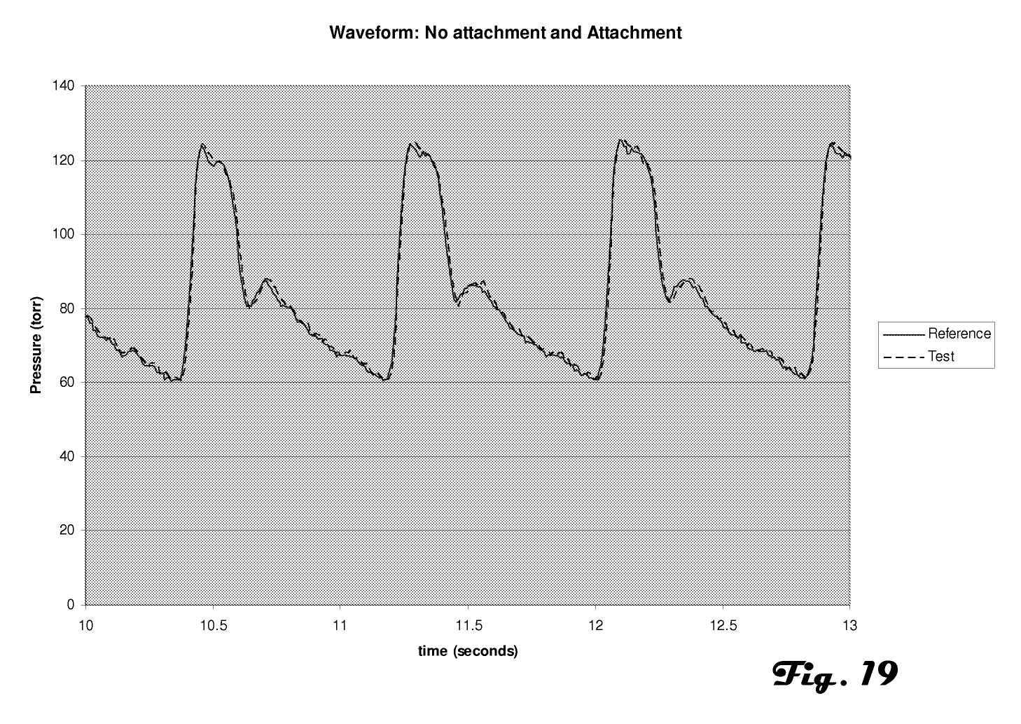

The realization of a system that can enable attachment of an automated blood measurement system to an existing hemodynamic monitoring system involved careful consideration of the circuit design and tubing selected. Variables that must be balanced correctly included tubing stiffness, tubing length, and tubing diameter. The AAMI document titled “Evaluation of Clinical Systems for Invasive Blood Pressure Monitoring” contains information on the influences of these variables. The impact of attaching an automated blood measurement device to the pressure monitoring system was evaluated. The same setup as described above was used but the stopcock was opened to allow fluid connectivity to both the pressure monitoring system and the automated glucose measurement system. Typically this is referred to as a 4-way stop-cock. Calculation of the natural frequency and damping resulted in values that are viewed as acceptable based on Gardner's wedge. The actual sweep test results were plotted on a standard Bode plot, FIG. 18. The Fig. shows test results from the pressure transducer alone and the condition where the automated blood measurement system is attached. However, a careful examination of the Bode plot shows a small resonance frequency peak at about 10 Hz automated blood measurement system. Additional waveform testing, FIG. 19, reveals no significant distortion the waveform or difference in the systolic pressure or diastolic pressure. In summary, the disclosed embodiment demonstrates the ability to attach an automated blood measurement system without creating pressure measurement errors.

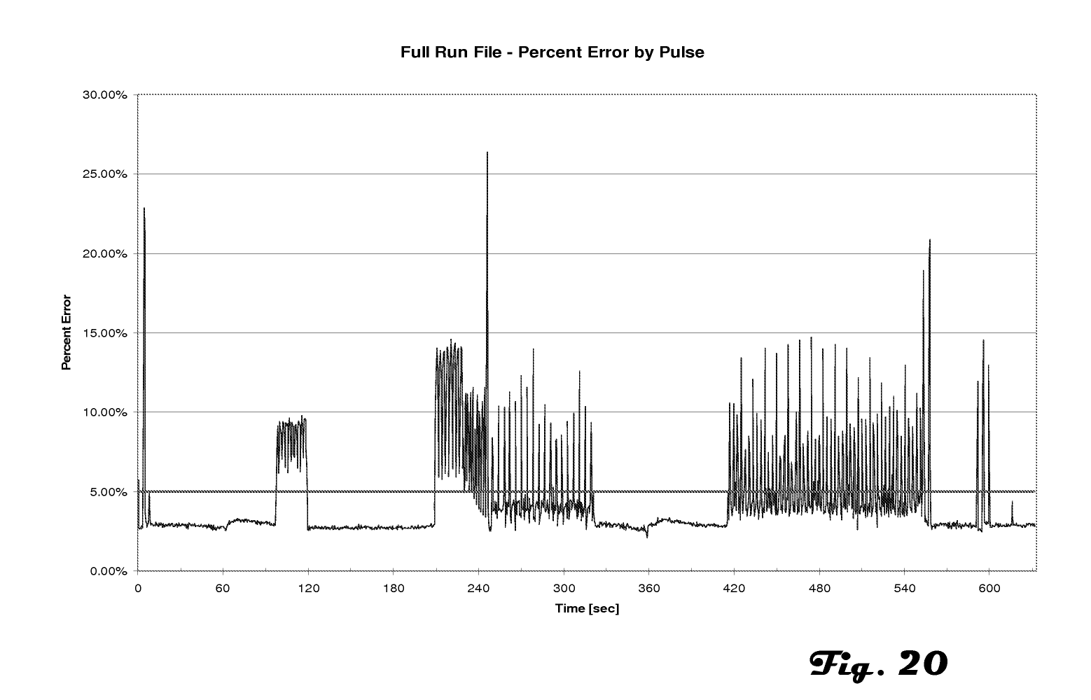

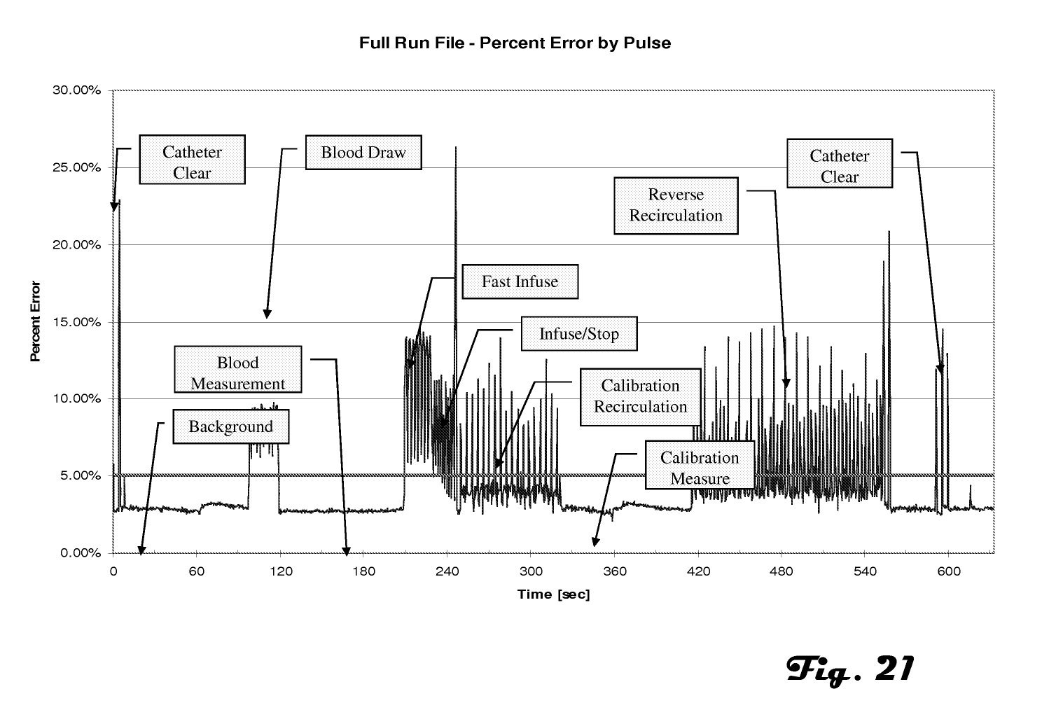

The impact of a measurement cycle on hemodynamic monitoring performance was determined. The variable pressure, variable volume system (aka the artificial patient) was attached as shown in FIG. 16. A standard blood measurement cycle was initiated and reference pressure transducer and test pressure transducer measurements recorded. The comparison of these measurements was done on a pulse by pulse basis. FIG. 20 shows the percent absolute error on a pulse by pulse basis for the entire measurement cycle. The solid line at 5% error enables easy visualization of the measurement cycle stages that create appreciable pressure measurement errors. FIG. 21 has each significant stage of the measurement cycle identified by name. The stages and their corresponding purpose are as follows:
a. Catheter Clear: an infusion pulse to clear catheter before draw
b. Background: a first calibration point at one glucose concentration
c. Blood draw: pulls blood in to the circuit
d. Blood measurement: the period over which a measurement is made
e. Fast infuse: a stage that infuses the blood into the patient
f. Infuse/stop: a stage that infuses blood into the patient but does so by infusing and stopping, a process that improves overall cleaning
g. Calibration recirculation: a combination phase involving cleaning of the circuit in the movement of a second calibration solution to the sensor.
h. Calibration measurement: a second calibration point at a second glucose concentration.
i. Reverse recirculation: a stage to remove the second calibration solution from the sensor.

These stages are included as a representative example of a measurement cycle but one of ordinary skill in the art would be aware of many potential variances relative to the above described measurement cycle.

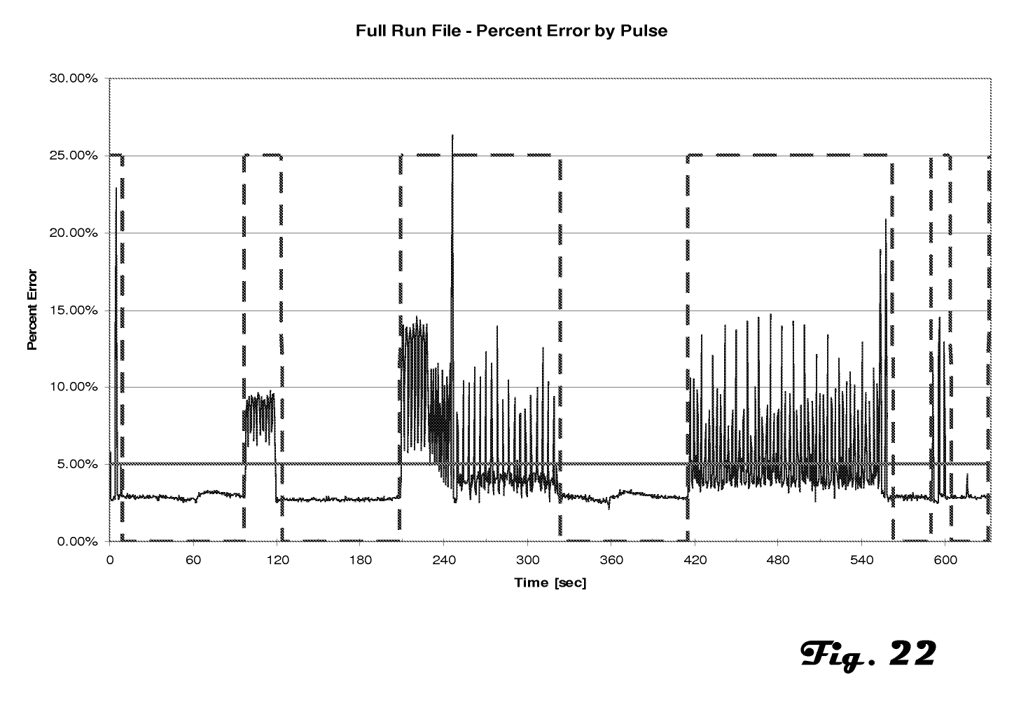

Examination of pulse-to-pulse error shows stages where all of the pulses exhibit a greater than 5% error while there are stages that exhibit intermittent errors greater than 5%. In practice, the display or use of any hemodynamic monitoring information obtained from a particular stage with either intermittent or continuous errors of greater than 5% should not be displayed to the clinician. The dotted line shown in FIG. 22 illustrates those areas of the measurement cycle where the hemodynamic monitoring information may be incorrect. As described previously, a surrogate signal can be displayed on the hemodynamic monitor during these periods. Additionally, the pressure artifacts introduced by the measurement cycle can be mitigated resulting in a continuous or near continuous monitoring profile as previously described.

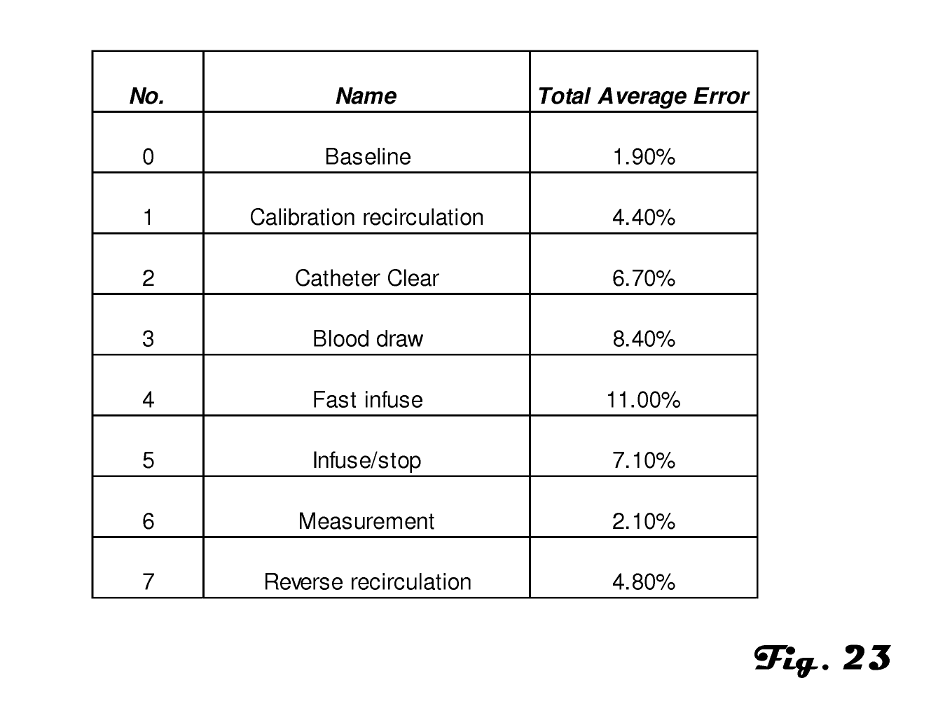

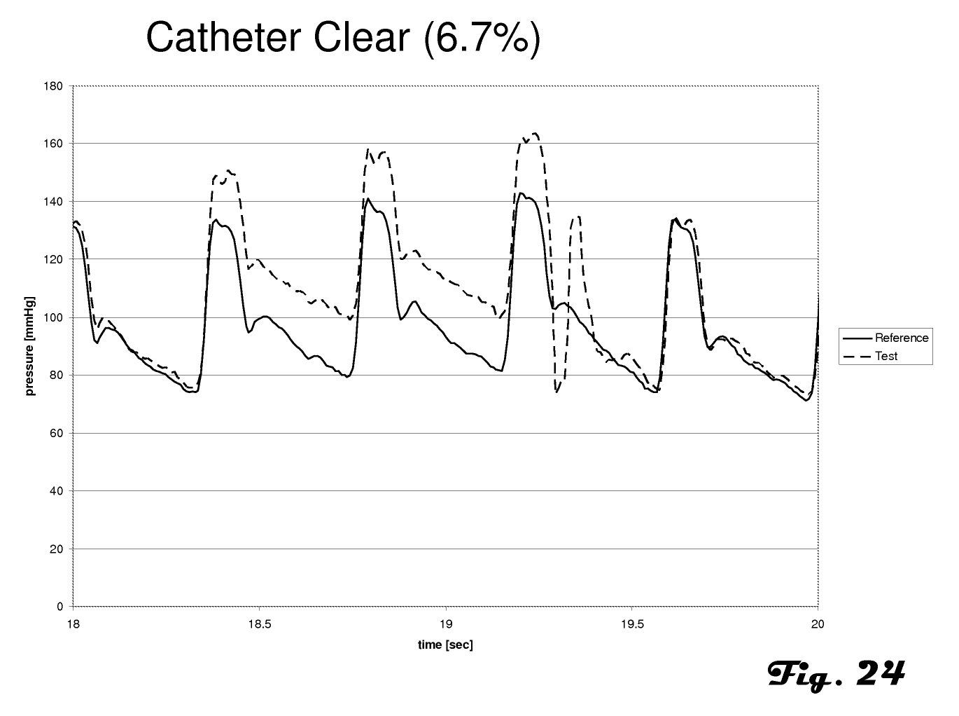

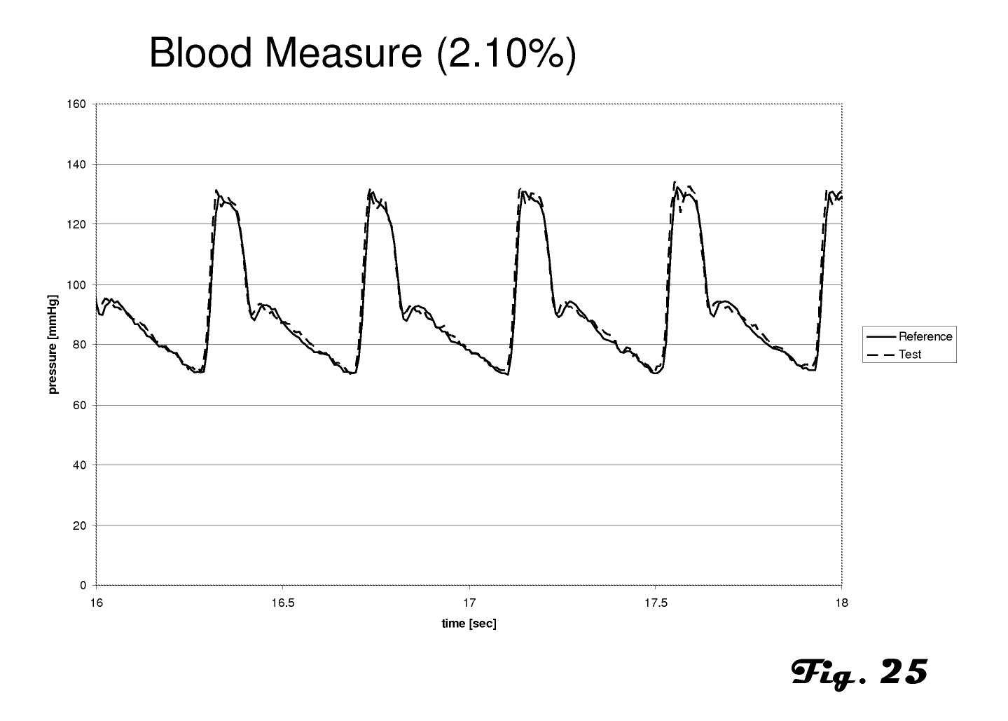

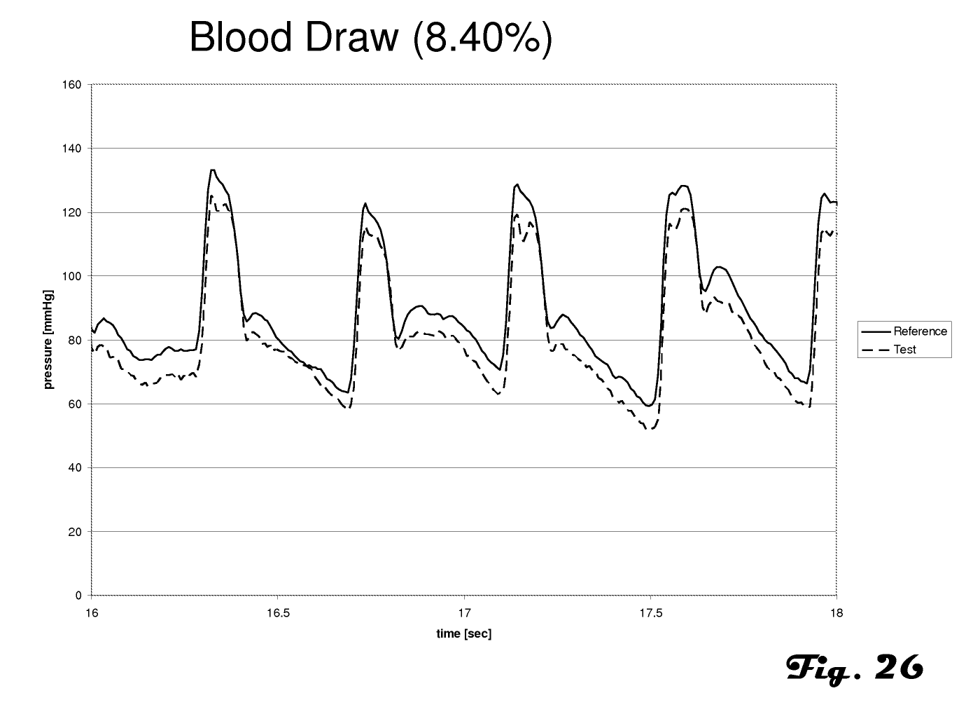

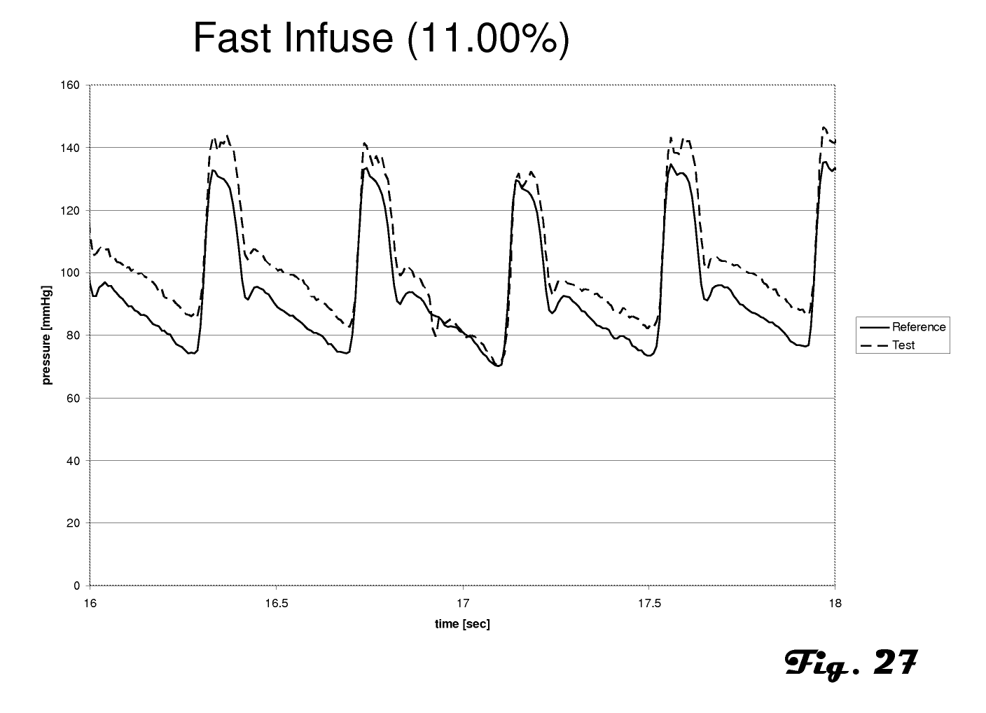

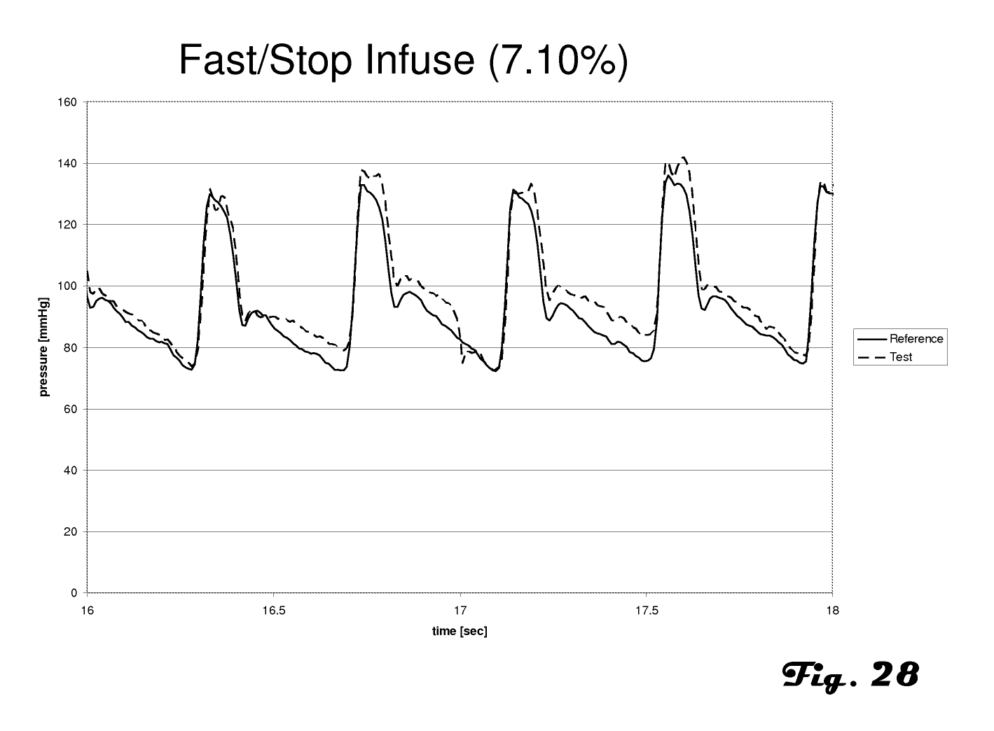

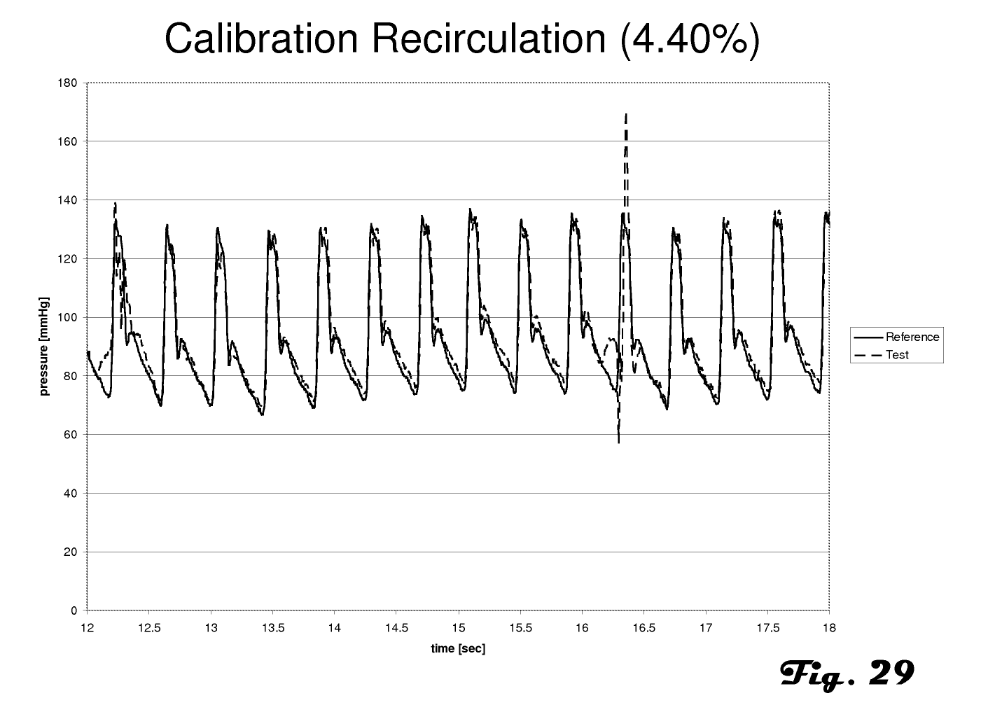

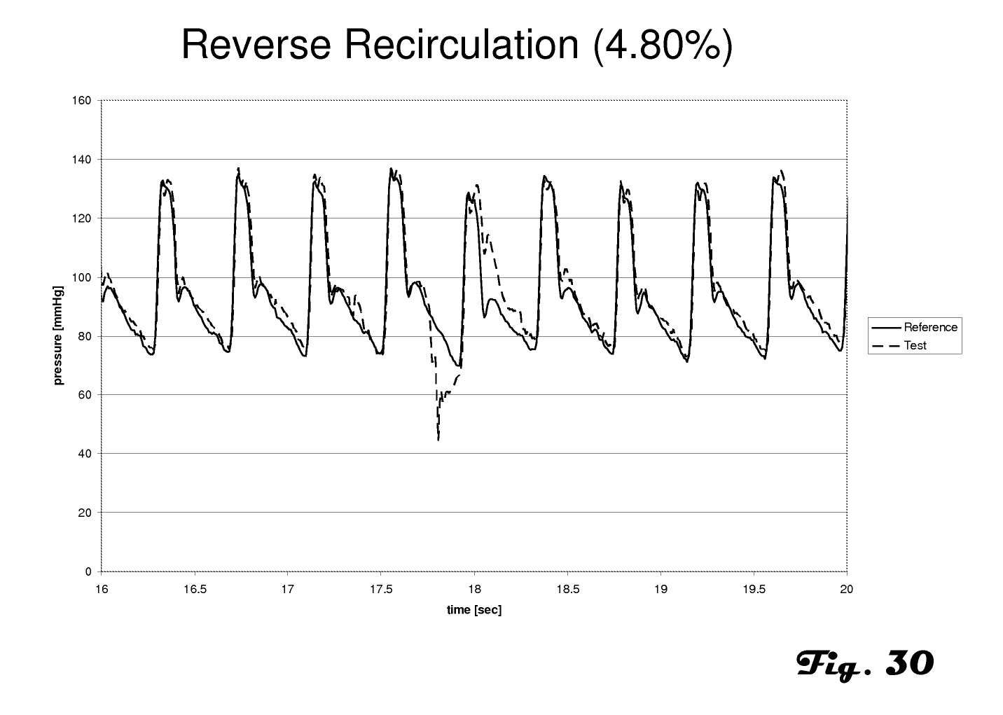

FIG. 23 quantifies the average error associated with each stage type. FIGS. 24 to 30 show the impact of the various stages on the hemodynamic monitoring. The type of pressure error is highly influenced by the type of flow used during the stage. For example, the blood draw stage uses a targeted pressure for control of the pump. The use of a pressure targeted flow leads to a relatively fixed offset that can be more easily compensated for then a more chaotic flow profile like the infuse/stop profile. FIG. 26 (blood draw) and FIG. 27 (fast infuse) are examples of pressure targeted flows there the majority of the error is an offset. The ability to correct for this type of error is relatively easy using either of the blood access systems shown in FIGS. 9 and 10. FIG. 29 (calibration recirculation) and FIG. 30 (recirculate saline) show relatively good agreement except for a single area of disruption. This area of disruption is caused by the peristaltic pump roller engaging or disengaging from the tube. The transient pressure signals that result are often referred to as roller events. The ability to correct for this type of error can be facilitated by the use of a different pumping mechanism, a peristaltic pump with more rollers, or by moving the pump slowly through these roller events. A re-examination of FIG. 20 would suggest that more than 50% of the time identified as having a hemodynamic monitoring error can be compensated for by direct offset compensation, careful management of the roller events, digital filtering, Kalman filter estimation, or a variety of other signal processing techniques. FIG. 24 (catheter clear) and FIG. 28 (fast/stop infuse) show a variety of pressure measurement errors and as such acceptable compensation may be more difficult.

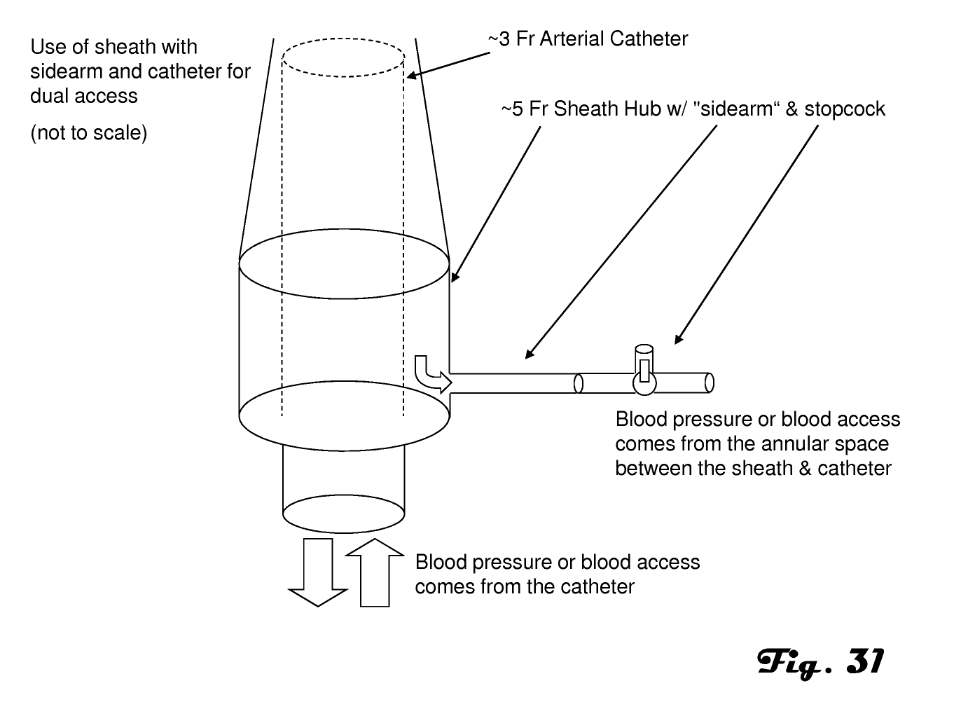

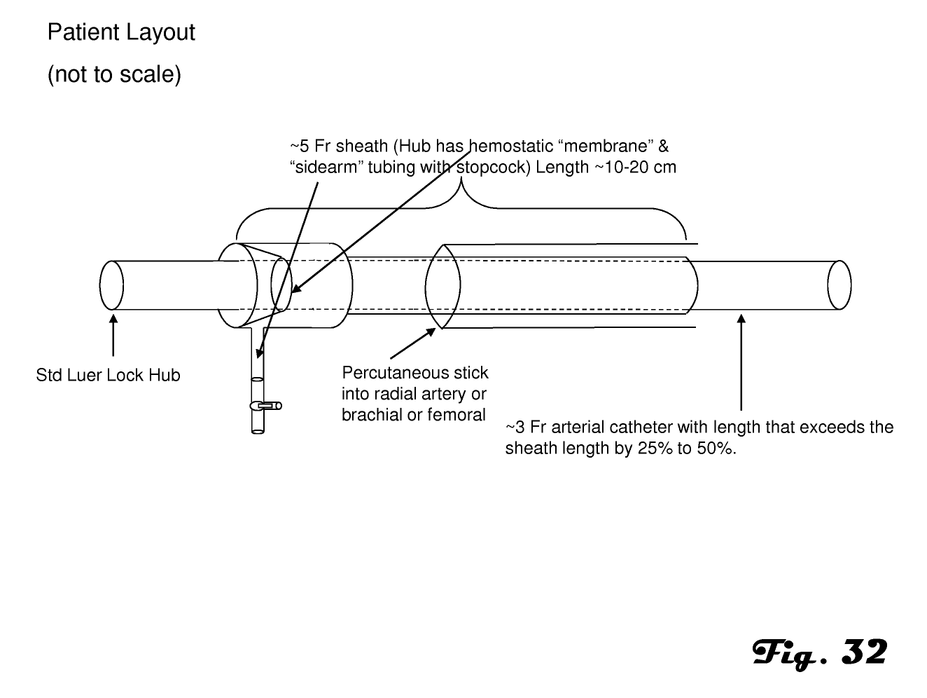

Many of the problems previously discussed are associated with the use of a single access site coupled with a single lumen catheter. Hemodynamic monitoring disruption can be mitigated by the use of an access mechanism that provided independent or semi-independent access through a single access location. For example a dual lumen catheter could be used. For example the Arrow International TWINCATH® 20/22 multiple-lumen peripheral catheter could be used in such a situation. The catheter contains two separate non-communicating lumens. Due to overall vessel compliance, the pressure gradient needed to infusion or removal several milliliters of blood over approximately a minute from an arterial access site will have no appreciable influence on the arterial pressure in the vessel. Another mechanism that provides access via two different pathways is the use of a arterial sheath with side arm and catheter. FIGS. 31-32 show an example embodiment of such a system. It is composed of a standard, off-the-sheath used in a variety of arterial-based interventional (radiology, cardiology, neuroradiology) procedures. The sidearm (with stopcock) of the sheath is integrated into the hub of the sheath. The hub typically contains a hemostasis membrane to minimize blood loss during the procedure. A smaller diameter arterial catheter is inserted thru the sheath into the artery. In use maintaining an ˜2 French difference between the sheath and catheter may be optimal for a good annular space. This annular space between the sheath and catheter can be used for blood draw by the automated blood measurement system or connected to the arterial pressure transducer. Correspondingly, the catheter can be used for attachment to the automated blood measurement system or connected to the arterial pressure transducer.

### EXAMPLE EMBODIMENTS

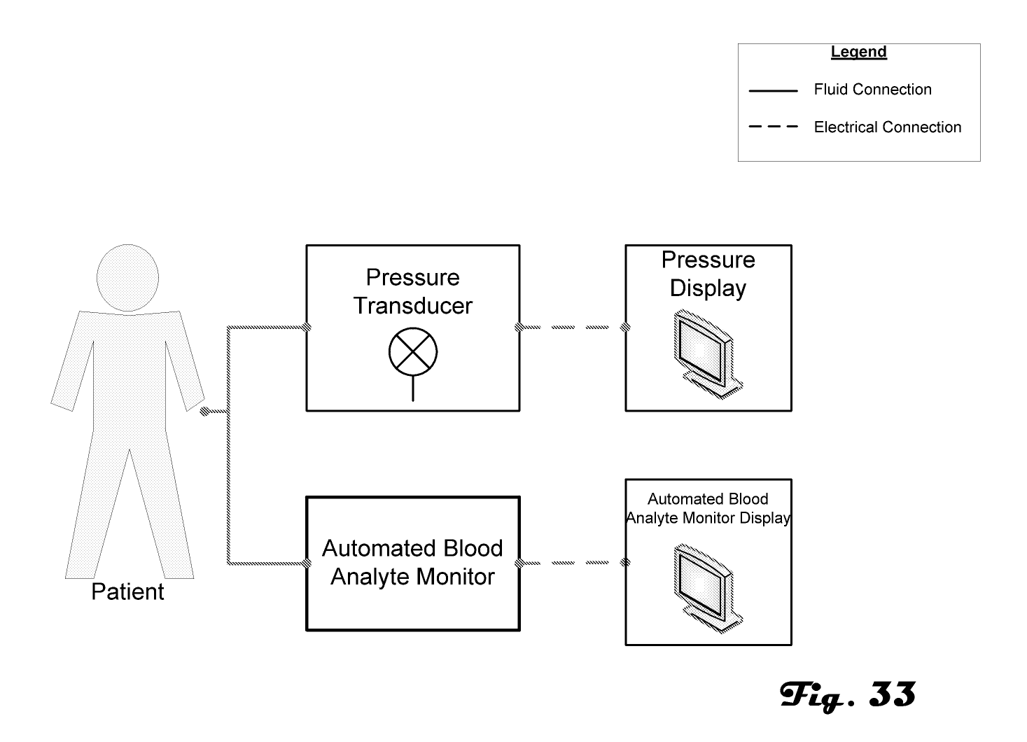

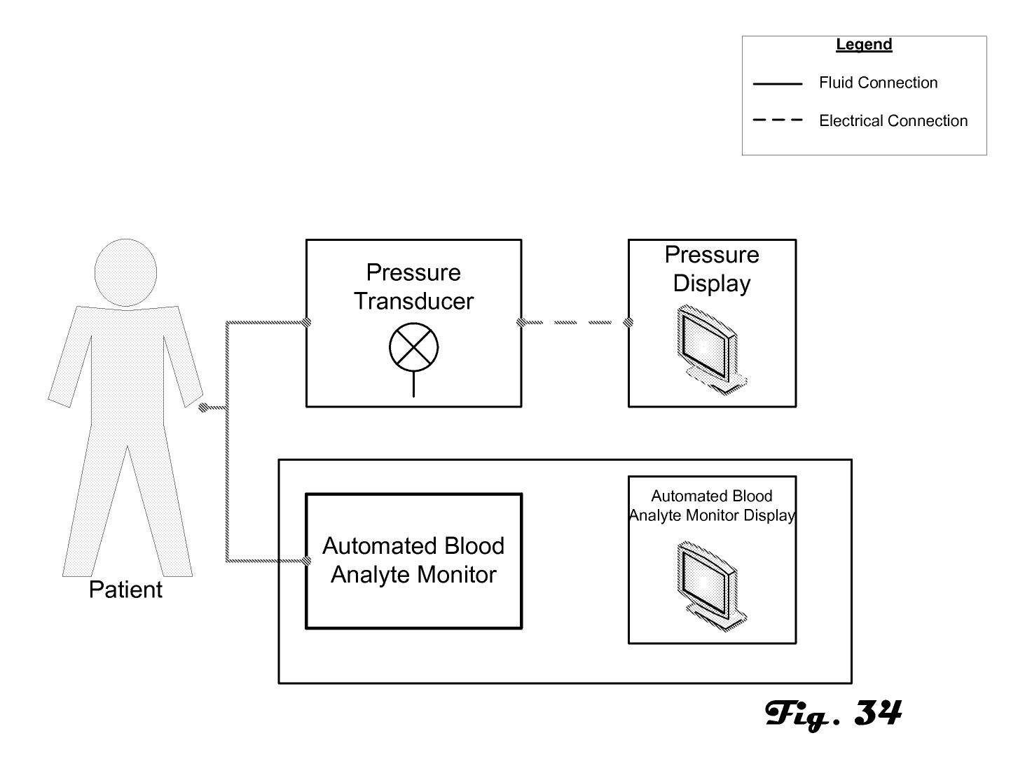

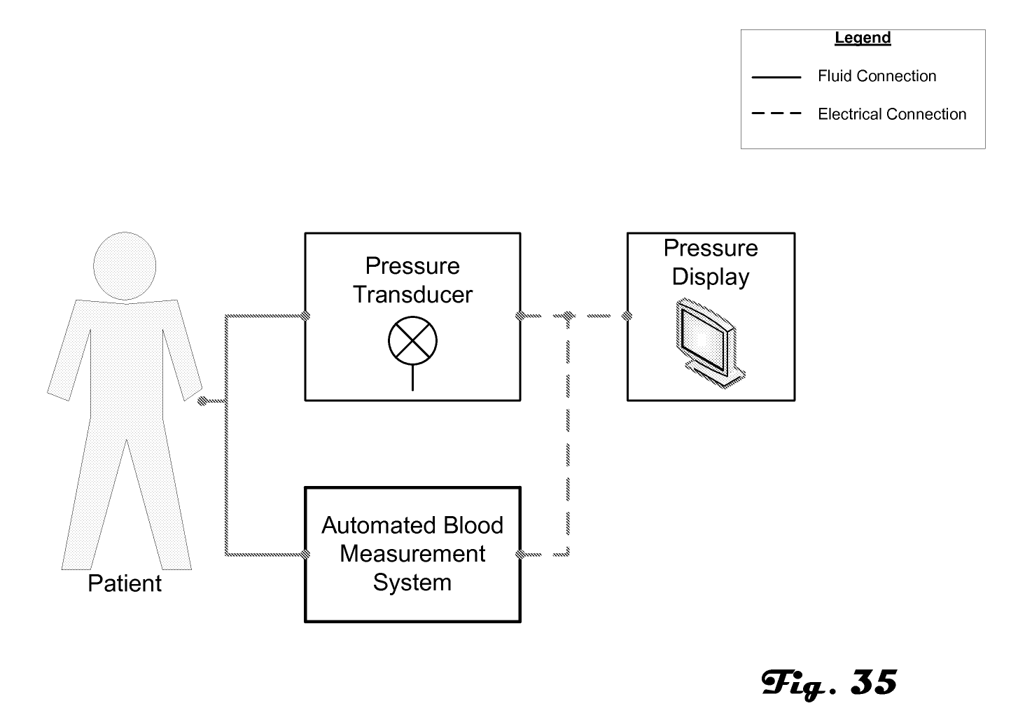

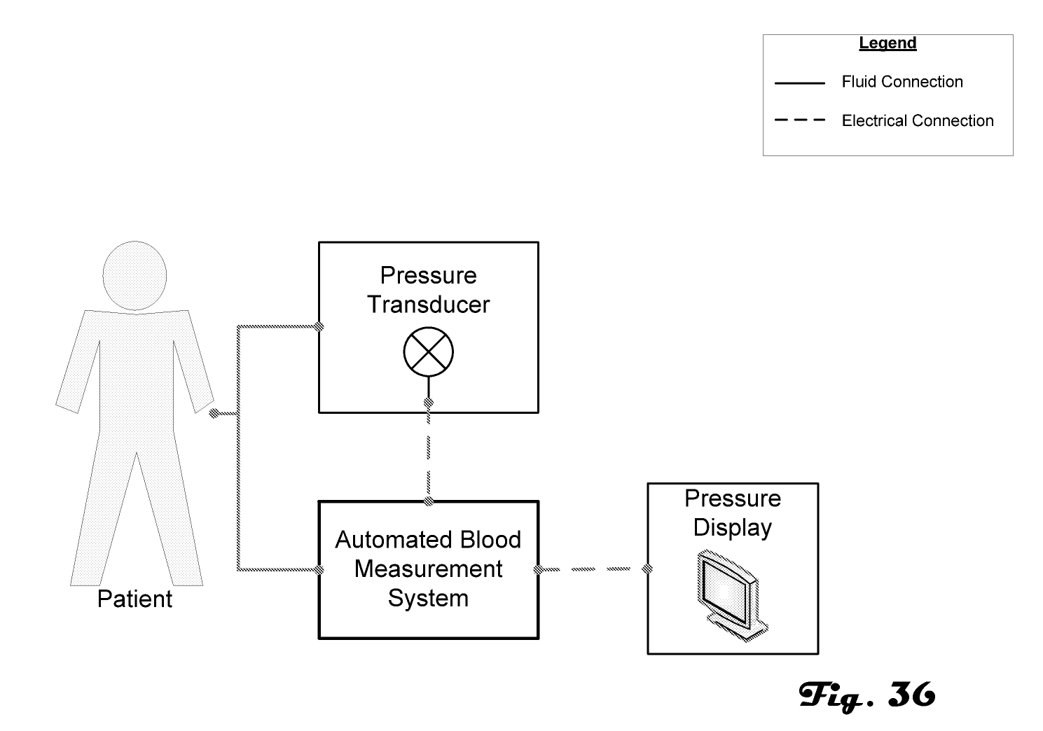

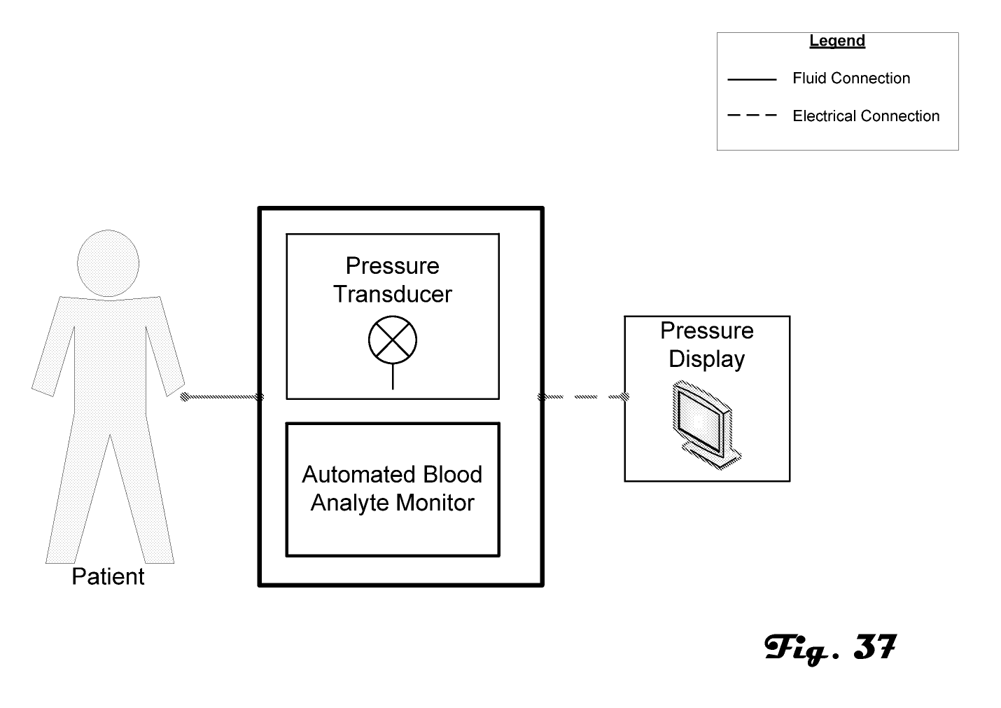

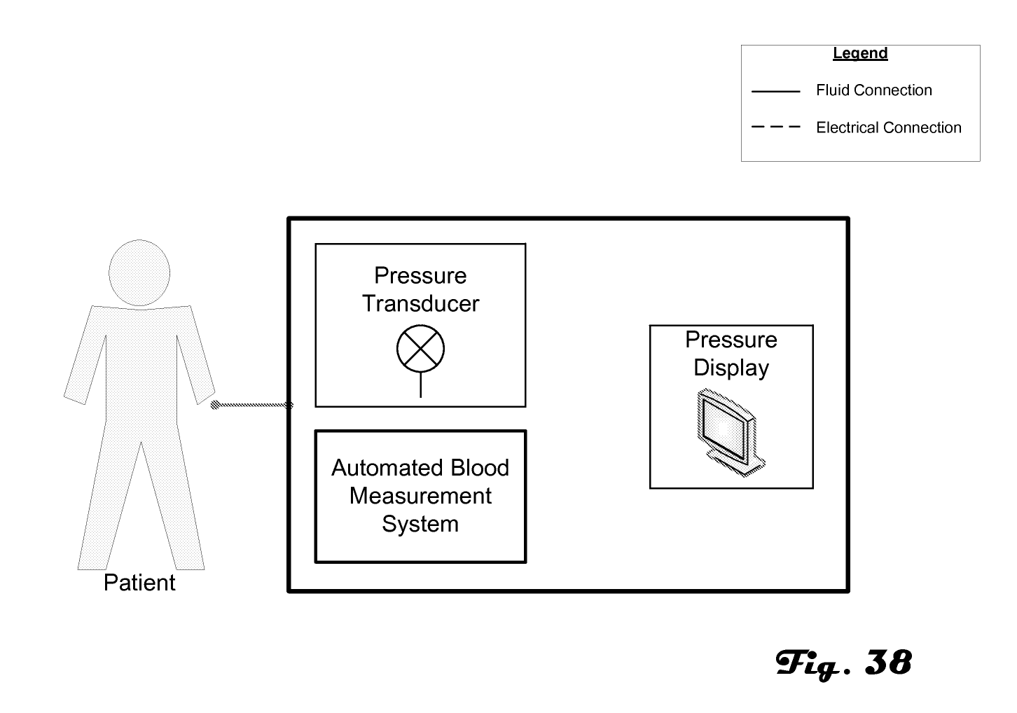

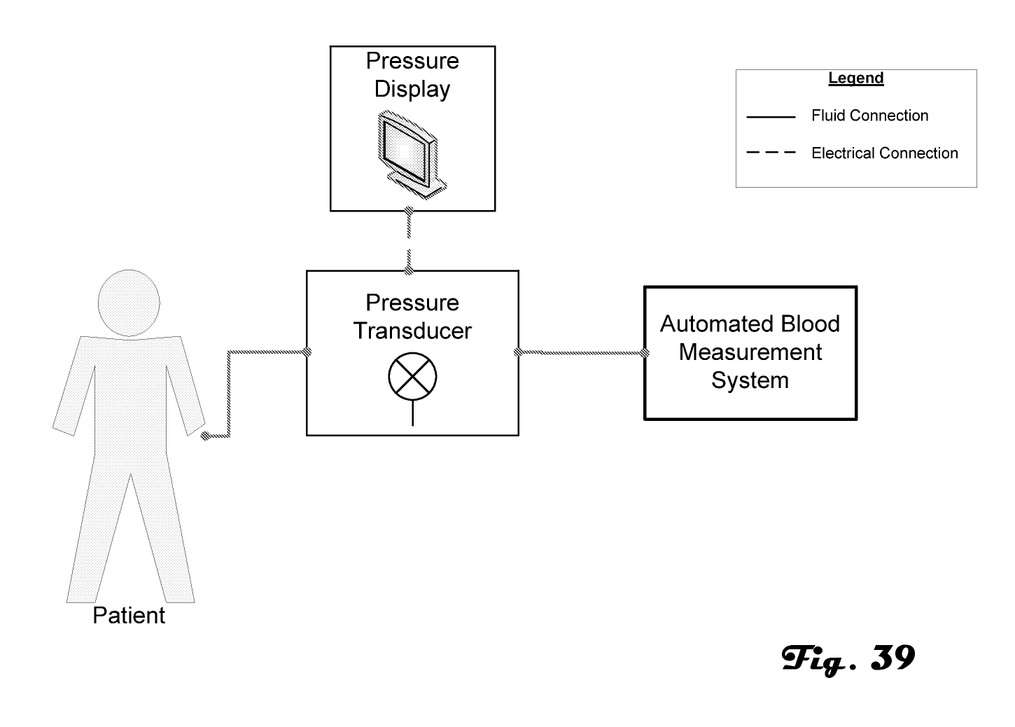

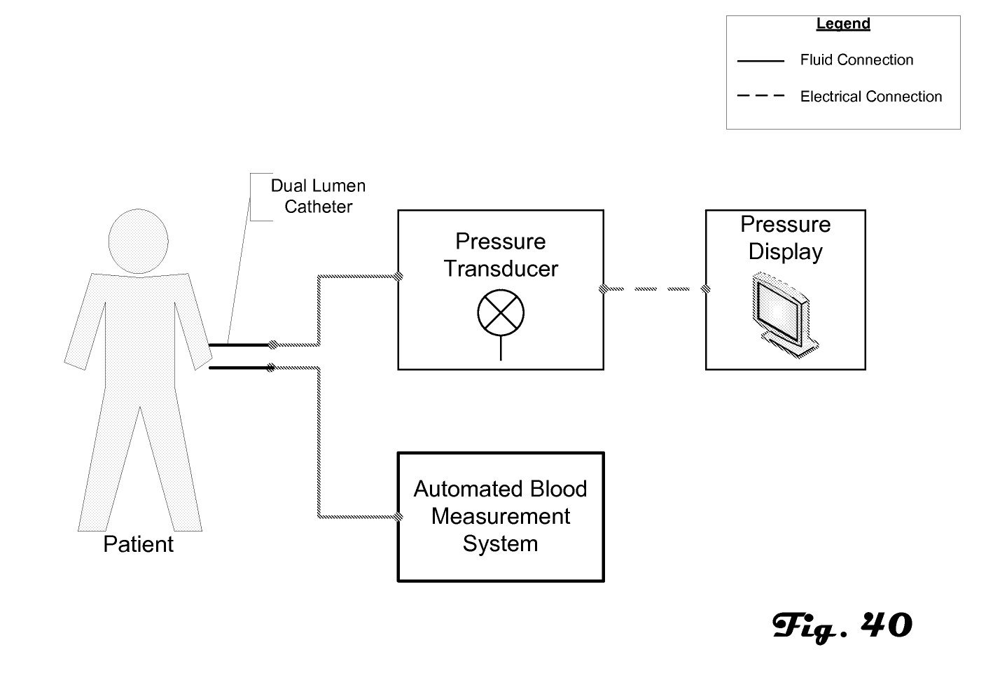

FIGS. 33 to 39 show a variety of configurations that satisfy the general objective of providing both hemodynamic monitoring as well as blood analyte measurements from a single access location. FIG. 33 illustrates a situation where the pressure transducer and the automated blood analyte system share a singular access site. No electrical connectivity is established between the pressure transducer and automated blood measurement system. Electrical connectivity exists between the automated blood analyte system and the automated blood analyte display. If hemodynamic monitoring disruption occurs then the automated blood analyte monitor display notifies the clinician via visual or audible alarms. FIG. 34 illustrates a situation where the pressure transducer and the automated blood analyte system share a singular access site. No electrical connectivity is established between the pressure transducer and automated blood measurement system. FIG. 35 illustrates a situation where the pressure transducer and the automated blood analyte system share a singular access site. Electrical connectivity is established between the pressure transducer, pressure display and automated blood measurement system. FIG. 36 illustrates a situation where the pressure transducer and automated blood measurement system share a single access site. Electrical connectivity exists between the pressure transducer and the automated blood measurement system. Electrical connectivity exists between the automated blood measurement system and the pressure display. FIG. 37 illustrates a situation where the pressure transducer and be automated blood measurement system exist within a single system. Electrical connectivity exists between the combined system and the pressure display. FIG. 38 illustrates a situation where the pressure transducer, automated blood measurement system, and pressure display exist within a single system. FIG. 39 illustrates a system with fluid connectivity between the patient and the pressure transducer. The automated blood measurement system is then in fluid connectivity with the pressure transducer. Electrical connectivity exists between the pressure transducer and the pressure display. FIG. 40 illustrates the use of a duel lumen catheter at a singular arterial access site. The pressure transducer and the automated blood measurement system are in direct fluid contact with the patient. The pressure transducer is electrically connected to the pressure display. Electrical connection between the automated blood measurement system and the pressure display is not shown but one of ordinary skill in the art would appreciate that this can occur.

An example apparatus according to the present invention comprises an arterial catheter, configured to be placed in fluid communication with an artery of a patient; a blood pressure monitoring subsystem mounted with the arterial catheter such that the blood pressure monitoring subsystem can determine the pressure of blood in the artery; and an analyte measuring subsystem mounted with the arterial catheter such that the analyte measuring subsystem can determine the presence, concentration, or both of one or more analytes in blood withdrawn from the artery.

An example apparatus according to the present invention comprises an arterial catheter, configured to be placed in fluid communication with an artery of a patient; a blood pressure monitoring subsystem mounted with the arterial catheter such that the blood pressure monitoring subsystem can determine the pressure of blood in the artery; and an analyte measuring subsystem mounted with the arterial catheter such that the analyte measuring subsystem can determine the presence, concentration, or both of one or more analytes in blood withdrawn from the artery. In such an example apparatus the arterial catheter can have first and second lumens, and the blood pressure measuring subsystem can be mounted in fluid communication with first lumen, and the analyte measuring subsystem can be mounted in fluid communication with the second lumen.

An example apparatus according to the present invention comprises an arterial catheter, configured to be placed in fluid communication with an artery of a patient; a blood pressure monitoring subsystem mounted with the arterial catheter such that the blood pressure monitoring subsystem can determine the pressure of blood in the artery; and an analyte measuring subsystem mounted with the arterial catheter such that the analyte measuring subsystem can determine the presence, concentration, or both of one or more analytes in blood withdrawn from the artery. In such an example apparatus, the arterial catheter can comprise (i) a hub defining an internal volume characterized by an internal diameter and having a fluid port in fluid communication with the internal volume; and (ii) a catheter having an external diameter less than the hub internal diameter and mounted within the internal volume; and the pressure monitoring subsystem can be mounted in fluid communication with either the fluid port of the hub or the catheter, and the analyte measuring subsystem can be mounted in fluid communication with the other of the fluid port of the hub or the catheter.

An example apparatus according to the present invention comprises an arterial catheter, configured to be placed in fluid communication with an artery of a patient; a blood pressure monitoring subsystem mounted with the arterial catheter such that the blood pressure monitoring subsystem can determine the pressure of blood in the artery; and an analyte measuring subsystem mounted with the arterial catheter such that the analyte measuring subsystem can determine the presence, concentration, or both of one or more analytes in blood withdrawn from the artery. In such an example apparatus, the analyte measuring subsystem can transport blood from the catheter; and the apparatus can further comprise an alarm and display subsystem, responsive to the blood pressure monitoring device and the analyte measuring subsystem, configured such that an alarm is indicated when both (i) the pressure monitoring subsystem indicates pressure outside a range of acceptable values and (ii) the analyte measuring subsystem indicates that the pressure monitoring subsystem indication is not invalidated by the analyte measuring subsystem.

In an example apparatus as in the preceding paragraph, the alarm and display subsystem can be further configured to display (i) an indication of pressure responsive to the pressure monitoring subsystem when the analyte measuring subsystem does not indicate interference with the pressure monitoring subsystem, and (ii) an indication that analyte measurement subsystem is interfering with the pressure monitoring subsystem when the analyte measuring subsystem does indicate interference with the pressure monitoring subsystem.

In an example apparatus as in the preceding paragraph, the indication that the analyte measurement subsystem is interfering with the pressure monitoring subsystem can comprise one or more of a text message, a change in color of the display, a change in size of a displayed waveform, or a waveform with a shape recognizably distinct from normal patient pressure waveforms.

An example apparatus according to the present invention comprises an arterial catheter, configured to be placed in fluid communication with an artery of a patient; a blood pressure monitoring subsystem mounted with the arterial catheter such that the blood pressure monitoring subsystem can determine the pressure of blood in the artery; and an analyte measuring subsystem mounted with the arterial catheter such that the analyte measuring subsystem can determine the presence, concentration, or both of one or more analytes in blood withdrawn from the artery. In such an example apparatus, the analyte measuring subsystem can transport blood from the catheter; and the apparatus can further comprise a display subsystem, responsive to the blood pressure monitoring device and the analyte measuring subsystem, configured to display a pressure indicated by the pressure monitoring subsystem when the analyte measuring subsystem is not interfering with the pressure measurement subsystem, and to determine and display a compensated pressure measurement during times when the analyte measurement subsystem is interfering with the pressure measurement subsystem.

In an example apparatus as in the preceding paragraph, the display subsystem can determine a compensated pressure measurement according to the output of the pressure sensor and information provided by the analyte measurement subsystem.

An example apparatus according to the present invention comprises an arterial catheter, configured to be placed in fluid communication with an artery of a patient; a blood pressure monitoring subsystem mounted with the arterial catheter such that the blood pressure monitoring subsystem can determine the pressure of blood in the artery; and an analyte measuring subsystem mounted with the arterial catheter such that the analyte measuring subsystem can determine the presence, concentration, or both of one or more analytes in blood withdrawn from the artery. In such an example apparatus, the mechanical compliance of the combination of the pressure monitoring subsystem and the analyte measuring subsystem satisfies the Gardner wedge criteria.

A method of calibrating any of the example apparatuses described herein can comprise operating the analyte measurement system such that fluid movement during calibration does not introduce errors of more than 5% in the output of the pressure monitoring subsystem.

An example apparatus according to the present invention comprises an arterial catheter, configured to be placed in fluid communication with an artery of a patient; a blood access subsystem, comprising: an analyte measurement device; a pressure sensor; a fluid path from the arterial catheter to the analyte measurement device and to the pressure sensor; at least one pump configured to move fluid in the fluid pathways; and a control system operatively connected to the pump to control operation of the pump; and a pressure determination system responsive to the pressure sensor and to the control system, configured to determine a signal corresponding to pressure in the artery from the pressure sensor and from the characteristics of the pump as indicated by the control system.

In an example apparatus as in the preceding paragraph, the pressure determination system can determine a signal corresponding to pressure in the artery by a lumped parameter model.

An example analyte measurement apparatus according to the present invention comprises a blood access subsystem, configured to transport fluid from a fluid access port connected to an arterial catheter during defined fluid transport times; an analyte measurement subsystem, configured to determine an analyte property of said withdrawn blood; and a pressure signal communication subsystem, configured to accept an input pressure signal from a pressure measurement system in fluid communication with the fluid access port, and to output a signal determined by (i) the input pressure signal except during fluid transport times, and (ii) a determined signal during fluid transport times.

In an example apparatus as in the preceding paragraph, the determined signal can correspond to a compensated pressure signal. In an example apparatus as in the preceding paragraph, the determined signal can comprise a signal having a high value, a low value, and a frequency similar to that of the input pressure signal during times that are not fluid communication times, but that has a waveform shape that is observably different from that of the input pressure signal during times that are not fluid transport times.

In an example apparatus as in the preceding paragraph, the waveform shape can comprise a square wave, a triangle wave, a simulated pressure wave with noise added, or a combination of any of two or more of the preceding.

Having thus described in detail certain embodiments of the present invention, it is to be understood that the invention described herein is not to be limited to particular details set forth in the above description as many apparent variations and equivalents thereof are possible without departing from the spirit or scope of the present invention.

We claim:

1. An apparatus for hemodynamic monitoring and analyte measurement, comprising:
a. An arterial catheter, configured to be placed in fluid communication with an artery of a patient;
b. A blood pressure monitoring subsystem mounted with the arterial catheter such that the blood pressure monitoring subsystem can determine the pressure of blood in the artery; and
c. An analyte measuring subsystem mounted with the arterial catheter such that the analyte measuring subsystem can determine the presence, concentration, or both of one or more analytes in blood withdrawn from the artery.

2. An apparatus as in claim 1, wherein the arterial catheter has first and second lumens, and wherein the blood pressure measuring subsystem is mounted in fluid communication with first lumen, and wherein the analyte measuring subsystem is mounted in fluid communication with the second lumen.

3. An apparatus as in claim 1, wherein the arterial catheter comprises (i) a hub defining an internal volume characterized by an internal diameter and having a fluid port in fluid communication with the internal volume; and (ii) a catheter having an external diameter less than the hub internal diameter and mounted within the internal volume; and wherein the pressure monitoring subsystem is mounted in fluid communication with either the fluid port of the hub or the catheter, and the analyte measuring subsystem is mounted in fluid communication with the other of the fluid port of the hub or the catheter.

4. An apparatus as in claim 1, wherein the analyte measuring subsystem can transport blood from the catheter; and further comprising an alarm and display subsystem, responsive to the blood pressure monitoring device and the analyte measuring subsystem, configured such that an alarm is indicated when both (i) the pressure monitoring subsystem indicates pressure outside a range of acceptable values and (ii) the analyte measuring subsystem indicates that the pressure monitoring subsystem indication is not invalidated by the analyte measuring subsystem.

5. An apparatus as in claim 4, wherein the alarm and display subsystem is further configured to display (i) an indication of pressure responsive to the pressure monitoring subsystem when the analyte measuring subsystem does not indicate interference with the pressure monitoring subsystem, and (ii) an indication that analyte measurement subsystem is interfering with the pressure monitoring subsystem when the analyte measuring subsystem does indicate interference with the pressure monitoring subsystem.

6. An apparatus as in claim 5, wherein the indication that the analyte measurement subsystem is interfering with the pressure monitoring subsystem comprises one or more of a text message, a change in color of the display, a change in size of a displayed waveform, or a waveform with a shape recognizably distinct from normal patient pressure waveforms.

7. An apparatus as in claim 1, wherein the analyte measuring subsystem can transport blood from the catheter; and further comprising a display subsystem, responsive to the blood pressure monitoring device and the analyte measuring subsystem, configured to display a pressure indicated by the pressure monitoring subsystem when the analyte measuring subsystem is not interfering with the pressure measurement subsystem, and to determine and display a compensated pressure measurement during times when the analyte measurement subsystem is interfering with the pressure measurement subsystem.

8. An apparatus as in claim 7, wherein the display subsystem determines a compensated pressure measurement according to the output of the pressure sensor and information provided by the analyte measurement subsystem.

9. An apparatus as in claim 1, wherein the mechanical compliance of the combination of the pressure monitoring subsystem and the analyte measuring subsystem satisfies the Gardner wedge criteria.

10. A method of calibrating the analyte subsystem of the apparatus of claim 1, comprising operating the analyte measurement system such that fluid movement during calibration does not introduce errors of more than 5% in the output of the pressure monitoring subsystem.

11. An apparatus for hemodynamic monitoring and analyte measurement, comprising:
a. An arterial catheter, configured to be placed in fluid communication with an artery of a patient;
b. A blood access subsystem, comprising: an analyte measurement device; a pressure sensor; a fluid path from the arterial catheter to the analyte measurement device and to the pressure sensor; at least one pump configured to move fluid in the fluid pathways; and a control system operatively connected to the pump to control operation of the pump; and
c. A pressure determination system responsive to the pressure sensor and to the control system, configured to determine a signal corresponding to pressure in the artery from the pressure sensor and from the characteristics of the pump as indicated by the control system.

12. An apparatus as in claim 11, wherein the pressure determination system determines a signal corresponding to pressure in the artery by a lumped parameter model.

13. An analyte measurement apparatus, comprising:
a. A blood access subsystem, configured to transport fluid from a fluid access port connected to an arterial catheter during defined fluid transport times;
b. An analyte measurement subsystem, configured to determine an analyte property of said withdrawn blood; and
c. A pressure signal communication subsystem, configured to accept an input pressure signal from a pressure measurement system in fluid communication with the fluid access port, and to output a signal determined by (i) the input pressure signal except during fluid transport times, and (ii) a determined signal during fluid transport times.

14. A system as in claim 13, wherein the determined signal corresponds to a compensated pressure signal.

15. A system as in claim 13, wherein the determined signal comprises a signal having a high value, a low value, and a frequency similar to that of the input pressure signal during times that are not fluid communication times, but that has a waveform shape that is observably different from that of the input pressure signal during times that are not fluid transport times.

16. An apparatus as in claim 15, wherein the waveform shape comprises a square wave, a triangle wave, a simulated pressure wave with noise added, or a combination of any of two or more of the preceding.
